(19) United States
(12) Patent Application Publication
(10) Pub. No.: US 2024/0219364 A1
(43) Pub. Date: Jul. 4, 2024

(54) SOIL SAMPLING METHODS AND SYSTEMS

(71) Applicants: Yard Stick PBC, Dover, DE (US); Soil Health Institute, Morrisville, NC (US)

(72) Inventors: Marissa Wiseman, Austin, TX (US); Michael Patrick Cope, Durham, NC (US); Cristine L.S. Morgan, Apex, NC (US); Jason Paul Ackerson, Lafayette, IN (US)

(73) Assignees: Yard Stick PBC, Dover, DE (US); Soil Health Institute, Morrisville, NC (US)

(21) Appl. No.: 18/397,718

(22) Filed: Dec. 27, 2023

Related U.S. Application Data
(60) Provisional application No. 63/435,699, filed on Dec. 28, 2022.

(51) Int. Cl. G01N 33/24; G06F 16/29

(52) U.S. Cl. CPC G01N 33/24; G06F 16/29; G01N 2033/245

(57) ABSTRACT

Methods and systems for soil sampling are generally provided. Systems and methods described herein may stratify a geographic region together using a clustering method and one or more soil properties to form strata. These strata may then be used to develop an appropriate sampling plan for measuring a desired soil property in the geographic region.

### CROSS-REFERENCE TO RELATED APPLICATIONS

This application claims the benefit under 35 U.S.C. § 119(e) of U.S. provisional application Ser. No. 63/435,699, filed Dec. 28, 2022, the disclosures of which are incorporated by reference in their entirety.

### FIELD

Disclosed embodiments are related to systems and methods for measuring soil properties.

### BACKGROUND

Bulk soil content is an important property of geographic regions for use in agriculture. But measuring soil content can be costly, time-consuming, and error-prone.

### SUMMARY

Methods and systems for soil sampling are generally provided. Systems and methods described herein may stratify a geographic region together using a clustering method and one or more soil properties to form strata. These strata may then be used to develop an appropriate sampling plan for measuring a desired soil property in the geographic region.

In one aspect, a method of determining bulk soil carbon content in a geographic region is provided. According to some embodiments, the method comprises: obtaining an initial dataset including at least one soil property related to bulk soil carbon content for the geographic region; clustering a plurality of tiles corresponding to subdivided portions of the geographic region based at least in part on the at least one soil property to form strata; determining measurement locations within the strata to be sampled to determine the bulk soil carbon content of the geographic region.

In another aspect, a soil measurement planning system is provided. According to some embodiments, the soil measurement planning system comprises: a processor configured to: obtain an initial dataset including at least one soil property related to bulk soil carbon content for the geographic region; cluster a plurality of tiles corresponding to subdivided portions of the geographic region based at least in part on the at least one soil property to form strata; determine measurement locations within the strata to be sampled to determine the bulk soil carbon content of the geographic region.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF DRAWINGS

The accompanying drawings are not intended to be drawn to scale. In the drawings, each identical or nearly identical component that is illustrated in various figures may be represented by a like numeral. For purposes of clarity, not every component may be labeled in every drawing. In the drawings:

FIG. 1A presents a schematic, top-view illustration of soil types within a field, according to some embodiments;

FIG. 1B presents a schematic, top-view illustration of a plurality of tiles subdividing a geographic region, according to some embodiments;

FIG. 1C presents a schematic, top-view illustration of a plurality of tiles subdividing a geographic region, according to some embodiments;

FIG. 1D presents a schematic, top-view illustration of a plurality of tiles subdividing a geographic region, with shading indicating an average value of a soil property, according to some embodiments;

FIG. 1E presents a schematic, top-view illustration of two overlaid pluralities of tiles subdividing a geographic region, according to some embodiments;

FIG. 1F presents a schematic, top-view illustration of two overlaid pluralities of tiles subdividing a geographic region, with shading indicating an average value of a soil property, according to some embodiments;

FIG. 1G presents a schematic, top-view illustration of a plurality of tiles subdividing a geographic region, with shading indicating an average value of a soil property, according to some embodiments;

FIG. 1H presents a schematic, top-view illustration of a stratified plurality of tiles subdividing a geographic region, according to some embodiments;

FIG. 1I presents a schematic, top-view illustration of a stratified plurality of tiles subdividing a geographic region and a plurality of measurement locations, according to some embodiments;

FIG. 1J presents a schematic, top-view illustration of a stratified plurality of tiles subdividing a geographic region and a plurality of measurement locations, according to some embodiments;

FIG. 2 presents a schematic illustration of a method, according to some embodiments;

FIG. 3 presents a schematic illustration of a method, according to some embodiments;

FIG. 4 presents a schematic illustration of a method, according to some embodiments;

FIG. 5 presents a schematic illustration of a method, according to some embodiments;

FIG. 6 presents a schematic representation of one embodiment of a computing device that may be configured to implement any of the methods disclosed herein;

FIG. 7A presents a schematic representation of a geographic region, according to some embodiments;

FIG. 7B presents a schematic representation of an average value of a soil property of a geographic region, according to some embodiments;

FIG. 7C presents a schematic representation of a standard deviation of a soil property of a geographic region, according to some embodiments;

FIG. 7D presents a schematic representation of a soil property of a geographic region, according to some embodiments; and

FIG. 7E presents a schematic representation of a stratification of a geographic region and a plurality of measurement locations, according to some embodiments.

### DETAILED DESCRIPTION

Bulk soil carbon content (e.g., bulk soil carbon density, or total carbon content of a geographic region) is a parameter that may be used for land management, and its accurate measurement is oftentimes desirable to guide agricultural decision making. However, measuring bulk soil carbon content can be cumbersome, requiring physical sampling of a geographic region in a variety of locations in order to develop a statistically robust estimate. Soil carbon is also rarely distributed uniformly throughout a field, and is related to a variety of other soil properties, such as the soil's bulk density, moisture content, clay content, and organic matter content, which can vary throughout a field-often discontinuously and/or unpredictably. The Inventors have recognized that this uncertainty in bulk soil carbon content makes it difficult to provide appropriate measurement plans for a geographic region while providing a set of measurements with a target accuracy. To address this many providers intentionally provide a large number of excess samples, but this may result in unnecessary costs and times. Therefore, the Inventors have recognized a desire to provide improved methods and systems for efficiently planning sampling of a geographic region for determining a desired soil property with a desired accuracy.

In view of the above, the Inventors have recognized the benefits associated with performing stratification of geographic regions by subdividing the geographic regions into tiles and clustering the tiles into strata with relatively homogenous bulk soil carbon density. The clustering methods provided herein may be used to identify a more optimal stratification of a geographic region for sampling purposes. By appropriately stratifying the various portions of s geographic region improved sampling plans may be developed, thereby reducing the total number of measurement locations at which the geographic region must be sampled in order to predict bulk soil carbon content, or other soil property, within a desired accuracy. This is in contrast to conventional stratification methods which typically stratify a geographic region into an arbitrary number of strata that may not accurately reflect the makeup of the geographic region and that may result in unnecessary increased sampling. Other types and benefits of systems and methods for bulk soil carbon content measurement are provided in greater detail below.

The process of measuring bulk soil carbon content may begin with an initial dataset. The initial dataset may provide at least one soil property related to bulk soil carbon content of a geographic region. For example, the data-set could provide actual values of a soil property, could provide a priori estimates of the soil property (e.g., based on a model of the soil property), combinations thereof, and/or any other appropriate type of dataset related to a desired soil property in a geographic region. Exemplary soil properties related to bulk soil carbon content may include, but are not limited to: bulk density, moisture content, clay content, and organic matter content. The exemplary soil property could, in fact, be an existing estimate of bulk soil carbon content (e.g., in a case where a more refined estimate of bulk soil carbon content is desired). The initial dataset may be or comprise a prediction based on a model of the geographic region produced using one or more geographic features of a geographic region, such as climate (e.g., annual rainfall, geographic region, daily sunlight, or latitude). In some embodiments, the initial dataset is at least partially informed by real measurements of the one or more soil properties (e.g., within or proximate the geographic region). The initial dataset could consist entirely of measured values (e.g., measured across a grid of locations), or could comprise modeled values based on a combination of real measurements and a model as described above. As a specific, non-limiting example, the initial dataset could come from the POLARIS dataset, a publicly available soil dataset including estimates of soil properties of soil in the United States.

Certain advantages have been recognized to using an initial dataset that comprises measured values of a soil property, as discussed above. For example, in some embodiments, it may be advantageous to measure one or more values of a soil property to generate an initial dataset. For example, the one or more values may be measured as part of a pre-sampling campaign. Although values from publicly available soil datasets such as POLARIS have advantages in terms of accessibility, it has been recognized that such publicly available datasets may be relatively inaccurate when describing soil properties, particularly within relatively small geographic regions. Without wishing to be bound by any particular theory, measured values may be used to supplement or replace publicly available initial data that might otherwise be relied upon in an initial dataset. Measured data may be measured by any of the measurement techniques described elsewhere herein, as the disclosure is not limited to any particular measurement technique for pre-sampled data used in an initial dataset. Furthermore, it should be noted that measurement locations for a pre-sampling campaign may be selected based on one or more of the methods provided herein, depending on the embodiment. For example, in some embodiments, a publicly available initial dataset may be used to perform a pre-sampling campaign according to a method (e.g., a method of determining bulk carbon) provided herein, and the results of the pre-sampling campaign may be used an initial dataset for a subsequent iteration of a method (e.g., a method of determining bulk carbon) provided herein. Other embodiments are also possible, as the disclosure is not so limited.

Any of a variety of appropriate methods may be used to obtain the initial dataset. For example, the initial dataset may be obtained, according to some embodiments by downloading the initial dataset from a remotely located server. In some embodiments, the initial dataset is obtained by recalling the initial dataset from non-transitory computer-readable memory, downloading the dataset from a remotely located database, manual entry, and/or any other appropriate method for obtaining the initial dataset. In some embodiments, the initial dataset is obtained from a preliminary set of measurements of the geographic region itself (e.g., which may be performed by a user of the method).

An initial dataset may include values of a soil property at a plurality of locations within a geographic region. For example, the initial dataset may include a plurality of estimates of average values of a soil property within a plurality of tiles (e.g., pixels) associated with the geographic region. In some cases, the initial dataset may also include a measure of statistical uncertainty within a tile, such as a standard-deviation, a variance, a quantile, a margin of error, or another measure of uncertainty in the soil property.

The methods and systems described herein may generally be used to determine sample measurement locations in any of a variety of types of geographic region. As used herein, the term geographic region refers to a continuous or discontinuous area or volume of land. In some embodiments, a geographic region consists of a single, continuous region. However, in some embodiments, the geographic region includes multiple, discontinuous regions. When the geographic region includes multiple, discontinuous regions, the discontinuous regions may be analyzed individually to determine appropriate measurement locations. However, depending on the embodiment, discontinuous regions of the geographic region may be analyzed simultaneously. The simultaneous analysis of discontinuous regions may, in some cases, advantageously reduce the total number of measurement locations needed to analyze the geographic region, relative to the number of measurement locations that would be needed to analyze the discontinuous regions separately. However, analyzing discontinuous regions together may be inappropriate if the geographic region includes large disparities in soil makeup. Thus, in some cases, separate analysis of the individual regions may be more advantageous. One of ordinary skill can choose a method appropriate to a geographic region to be analyzed.

In some embodiments, a geographic region may be modeled using an area model, wherein a soil property is assumed to be uniform as a function of depth within a region. Alternatively, a geographic region may be modeled using a volumetric model, wherein depth-variation of the soil within the geographic region is accounted for and measured. According to some embodiments, the area model, wherein depth is ignored, may be advantageous because variation in a soil property is comparatively low as a function of the soil's depth within the geographic region.

A geographic region may be subdivided using a plurality of tiles, according to some embodiments. The tiles may be two dimensional tiles (e.g., pixels) if the geographic region is an area. Alternatively, the tiles may be three dimensional tiles (e.g., voxels) if the geographic region is a volume. Tiles may be regular or irregular, depending on the embodiment. The tiles may subdivide the geographic region perfectly, such that no portion of a tile extends beyond the boundaries of the geographic region. However, in some embodiments, the plurality of tiles may subdivide the region imperfectly, such that they approximately cover the geographic region, but some tiles overlap with a boundary of the geographic region, some tiles extend beyond a boundary of the geographic region, and/or some portions of the geographic region are not perfectly covered by tiles. In other words, the tiles need not perfectly subdivide the geographic region, although of course they may, as the disclosure is not so limited. However, in some embodiments, the plurality of tiles may be non-overlapping tiles.

An estimate of a soil property, obtained from an initial dataset, may be used to estimate the value of the soil property at a plurality of tiles (e.g., a first plurality of tiles) subdividing a geographic region. The initial dataset may also subdivide the geographic region into a second plurality of tiles smaller than the first plurality of tiles, and include data associated with each tile of the second plurality of tiles. According to some embodiments, a soil property for tiles of the second plurality of tiles may be estimated using the values of the soil property associated with tiles of the first plurality of tiles, as discussed in greater detail below.

An estimate of a soil property at a plurality of tiles representing a geographic region may, in some embodiments, permit the stratification of the geographic region. Stratification may significantly reduce the variance in the measurement of bulk soil carbon content (e.g., relative to a bulk soil carbon content estimate obtained by randomly sampling the entire region to determine bulk soil carbon content). Stratification of a geographic region may generally refer to the classification of various portions of the geographic region into one or more strata. Each stratum of a geographic region has a set of soil properties (e.g., bulk soil carbon content, bulk density, moisture content, organic matter content, clay content) that are similar, and in some instances substantially equal, to other portions of the geographic region belonging to the stratum. For example, if a first tile within the geographic region and a second tile within the geographic region both belong to the same stratum, they may exhibit similar soil properties, according to some embodiments. Likewise, a stratum may be associated with a variance in each soil property that may be assumed to apply uniformly to the stratum.

In some embodiments, the average value of a soil property within a geographic region may be the weighted average of the soil property within the geographic region, weighted by a relative weight of the strata. For example, in some embodiments, the average value of a soil property within a geographic region can be computed using Eq. (1) below, where x_{gr} represents the average value of the property within the geographic region, n represents the total number of strata, xi represents the average value of the soil property (e.g., bulk soil carbon content) for the i^{th} stratum, and wᵢ represents the fraction of the geographic region occupied by i^{th} stratum (the weight of the i^{th} stratum).

$${x}_{gr} = {\sum }_{i = 1}^{n} {x}_{i} {w}_{i} \qquad ( 1 )$$

Of course other ways of calculating an average soil property may also be used as the disclosure is not limited to only using the above described methods.

Without wishing to be bound by any particular theory, obtaining a bulk soil carbon content estimate by randomly sampling the entire region would be equivalent to stratification using exactly one stratum. Thus, stratification is expected to decrease uncertainty (e.g., variance) relative to randomly sampling the entire region.

In some embodiments, variance in the carbon, or other soil property, of each stratum is included in the initial dataset. A variance expected for a bulk soil carbon content of the geographic region may be estimated, in some embodiments, by combining the variance in the bulk soil carbon content of the individual strata. However, the initial dataset need not provide this variance, and need not expressly provide the bulk soil carbon content of the individual strata. For example, in some embodiments, bulk soil carbon content (and an associated uncertainty) of the strata can be estimated based on values and/or variabilities of one or more of the soil properties provided in the initial dataset other than bulk soil carbon content. For example, in some embodiments, uncertainty in bulk soil carbon content may be estimated from uncertainty in organic matter content, because organic matter may be correlated with the carbon content in the soil.

Without wishing to be bound by any particular theory, there is no “optimal” method for stratifying a geographic region into strata. For example, stratification may be performed using the cumulative root frequency method (the “cum root f” rule), or any of a variety of other appropriate methods. However, such implementations can have drawbacks for the determination of bulk soil carbon content. In particular, without a priori knowledge of how many strata to use, such methods may result in sub-optimal stratification that significantly increases the number of measurement locations at which a geographic region must be sampled to determine the average bulk soil carbon content to within a target uncertainty. Thus, as elaborated on further below, different numbers of strata for grouping of the different portions of a geographic region may be used to determine appropriate sampling plans for comparison with one another to determine which plan provides a more optimized sampling plan.

In the context of the present disclosure, it has been recognized that clustering of tiles may be particularly advantageous for the stratification of a geographic region. A clustering algorithm may be used to classify individual tiles (e.g., pixels) corresponding to subdivided portions of the geographic region into strata, based on at least one soil property. If a desired number of strata is known a priori, the clustering algorithm may be used to cluster the tiles into the desired number of strata immediately. However, in some embodiments, the desired number of strata is unknown. Accordingly, it has been recognized that in some embodiments, it may be advantageous to perform the clustering step for a range of different numbers of strata. Clustering for a range of different numbers of strata may produce a plurality of groups of strata, wherein each group includes a different number of strata. Then, an appropriate number of strata can be chosen based on the demands of the situation and/or a comparison of the different sampling plans developed using these different groups of strata as elaborated on below.

It may be desirable to minimize a number of measurement points for measuring bulk soil carbon content in a geographic region for a provided target uncertainty (e.g., a target variance or an associated target standard error, target margin of error, or other target measure of uncertainty). Minimizing the number of measurement points may be particularly desirable in the context of bulk soil carbon content determination, because bulk soil carbon content can be expensive and time-consuming to measure accurately. In some embodiments, a target uncertainty is obtained. The target uncertainty may be user prescribed, or may be a default value. The geographic region may be stratified into a plurality of groups of strata, wherein each group includes a different number of strata. For the groups of strata (e.g., for each group of strata), according to some embodiments, a minimum number of measurement locations can be determined. The minimum number of measurement locations for a group of strata is, according to some embodiments, based on the constraint that the uncertainty in measured bulk soil carbon content should fall within the target uncertainty, and may be determined by allocating measurement locations to the strata and predicting the effect of the sampling at those measurement locations on the expected measurement uncertainty, as discussed in greater detail below.

When a minimum number of measurement locations for the groups of strata (e.g., for each group of strata) has been determined, a group of strata may be identified with the smallest minimum number of measurement locations. In some embodiments, this grouping of strata may be used to determine bulk soil carbon content, e.g., by actually determining appropriate measurement locations for the strata of the identified group of strata. Of course, it should be understood that in some cases the smallest minimum number of measurement locations need not be unique (e.g., different groups of strata may have an identical smallest number of measurement locations). In such cases, one of the groups of strata giving rise to the smallest number of measurement locations may be used. In such cases, the group of strata may be selected to minimize expected uncertainty, to minimize the number of strata, at random, or for any of a variety of other reasons.

According to some embodiments, the method comprises scaling the smallest minimum number of measurement locations by a multiplier to determine a scaled number of measurement locations. For example, if the smallest minimum number of measurement locations for stratification of a geographic region is 100, this value could be scaled by the multiplier 1.2 to give a total number of measurement locations of 120. The scaled number of measurement locations may then be allocated to the strata rather than the smallest minimum number of measurement locations, depending on the embodiment.

Any of a variety of suitable multipliers may be used to produce a scaled number of measurement locations. In some embodiments, a multiplier used to produce a scaled number of measurements is greater than to 1, greater than or equal to 1.5, greater than or equal to 2, greater than or equal to 2.5, greater than or equal to 3, greater than or equal to 3.5, greater than or equal to 4, or greater than or equal to 4.5. In some embodiments, a multiplier used to produce a scaled number of measurements is less than or equal to 5, less than or equal to 4.5, less than or equal to 4, less than or equal to 3.5, less than or equal to 3, less than or equal to 2.5, less than or equal to 2, or less than or equal to 1.5. Combinations of these ranges are also possible (e.g., greater than 1 and less than or equal to 5). Other ranges, both higher and lower than those described above, are also possible, as the disclosure is not so limited.

Alternatively, in some embodiments, a restriction on the number of measurement locations is imposed (e.g., by a cost constraint), and it may be desirable to obtain a target number of measurement locations, and to minimize the expected uncertainty (e.g., margin of error) associated with those measurement locations. In some embodiments, a target number of measurement locations may be obtained (e.g., may be user supplied, or may take a default value). The geographic region may be stratified into a plurality of groups of strata, wherein each group includes a different number of strata. For the groups of strata (e.g., for each group of strata), according to some embodiments, minimum uncertainties (e.g., variances or standard errors) of the bulk soil carbon content can be determined by allocating the target number of measurement locations to the strata, as discussed in greater detail below.

When the minimum uncertainties for the groups of strata (e.g., for each group of strata) have been determined, a group of strata may be identified that has the smallest minimum uncertainty. A method may comprise actually determining appropriate measurement locations for the strata of the identified group of strata. In some embodiments, the grouping of strata may be used to determine bulk soil carbon content.

Combinations of the above approaches are also possible and are expanded on further in reference to the figures below. For example, in some embodiments, both a target uncertainty and a target number of measurement locations may be provided (e.g., by assigning fixing the confidence of the model to a prescribed value). The geographic region may be stratified into a plurality of groups of strata, wherein each group includes a different number of strata. For the groups of strata (e.g., for each group of strata), according to some embodiments, minimum uncertainty of the bulk soil carbon content can be determined by allocating the target number of measurement locations to the strata, and the minimum number of measurement locations required to achieve a target uncertainty may be determined. If any group of strata achieves the target uncertainty, a group of strata achieving the target uncertainty and having a smallest number of measurement locations may be chosen. Conversely, if no group of strata achieves the target uncertainty, a group of strata may be identified that minimizes measurement uncertainty for the target number of measurement locations, and this group of strata may be chosen. Other combinations of method steps are also possible, as the disclosure is not so limited.

In some embodiments, after a stratification has been made using one of the above-mentioned methods, measurement locations may be selectively added to or removed from the strata in a way that minimizes target uncertainty. This may permit the number of measurement points to be selectively increased or decreased while maintaining an efficient allocation of measurement points between the strata. For example, if attaining a target uncertainty requires too many measurement points to be efficiently sampled, the change in uncertainty associated with excluding each measurement point can be determined, and the measurement point with the least impact on uncertainty can be removed. Such a process can be iterated until a sufficiently small number of measurement points is allocated. As an alternative example, an additional measurement point may be allocated by identifying a change in expected uncertainty associated with adding the measurement point to each stratum, and adding the measurement point to the stratum where addition of the measurement point causes the biggest reduction in expected uncertainty.

After stratification of the geographic region, according to some embodiments, appropriate measurement locations are determined. The measurement locations may be determined by any of a variety of appropriate methods. It may be advantageous, according to some embodiments, to first determine an appropriate number of measurement locations for each stratum (e.g., an allocation of the measurement locations between strata). In some embodiments, a Neyman allocation may be used to determine the measurement locations (e.g., by allocating measurement locations to the strata, so that the measurement locations can subsequently be selected from within the strata). Without wishing to be bound by any particular theory, for a given number of measurement locations (n) of the geographic region, a Neyman allocation may provide an allocation of those measurement locations between the strata of the geographic region that minimizes the expected uncertainty in the bulk soil carbon content. Generally, the number of measurement locations associated with the k^{th} stratum in an ideal Neyman allocation (nₖ) is given by Eq. (2):

$${n}_{k} = n \frac{{w}_{k} {σ}_{k}}{{\sum }_{j = 1}^{L} {w}_{j} {σ}_{j}} \qquad ( 2 )$$

where wₖ and wⱼ represent a fraction of a geographic region occupied by the k^{th} and j^{th} stratum respectively (e.g., an area fraction or a volume fraction), σₖ and σⱼ represent the square root of the variance of the bulk soil carbon content within the k^{th} and j^{th} stratum respectively, and L represents the total number of strata. In the case of stratifying regularly shaped tiles, this equation can be rewritten in the form of Eq. (3), where Nₖ and Nⱼ represent the number of tiles in the k^{th} and j^{th} stratum, respectively.

$${n}_{k} = n \frac{{N}_{k} {σ}_{k}}{{\sum }_{j = 1}^{L} {N}_{j} {σ}_{j}} \qquad ( 3 )$$

The Neyman allocation thus weights the sampling of each stratum by the overall size of the stratum and by the variance of the stratum, reducing sampling within uniform and small strata, according to some embodiments.

The variance (V) associated with a Neyman allocation stratifying regularly shaped tiles may be determined using Eq. (4) below:

$$V = {\sum }_{k = 1}^{L} ( 1 - \frac{{n}_{k}}{{N}_{k}} ) \frac{{N}_{k}^{2} {σ}_{k}^{2}}{{n}_{k}} \qquad ( 4 )$$

The associated standard error, (SE) can be calculated by Eq. (5) below, and the associated margin of error (e) for a desired confidence level δ_{α/2} can be calculated using Eq. (6).

$$SE = \sqrt{V} \qquad ( 5 )$$

$$e = {δ}_{\frac{α}{2}} \times SE \qquad ( 6 )$$

Of course, since without wishing to be bound by any particular theory, the Neyman allocation may merely approximate the allocation described by Eq. (2) or Eq. (3). For example, the nₖ values must take integer values and are subject to the constraint that n equals the total of all the nₖ values, whereas Eq. (2) and Eq. (3) allow nₖ to have a non-integer value. A Neyman allocation may be rounded, in some embodiments, such the number of measurement locations within each stratum is an integer. Without wishing to be bound by any particular theory, in some embodiments, similarity of a given allocation of measurement locations to an ideal Neyman allocation of the same number of measurement locations may be quantified by representing the allocation as a first vector (v_{All}) as shown in Eq. (7)

$${v}_{All} = ( {n}_{1} , {n}_{2} , … , {n}_{L} ) \qquad ( 7 )$$

where each nₖ refers to the number of measurement points allocated to the k^{th} stratum, and by representing the ideal Neyman allocation as a second vector (v_{Ney}) as shown in Eq. (8)

$${v}_{Ney} = ( {n}_{{Ney}_{1}} , {n}_{{Ney}_{2}} , … , {n}_{{Ney}_{L}} ) \qquad ( 8 )$$

where each n_{Neyk} refers to the number of measurement points allocated to the k^{th} stratum in the ideal Neyman allocation. The similarity score (S_{Ney}) of the allocation to an ideal Neyman allocation may then be quantified by Eq (9)

$${S}_{Ney} = \frac{{v}_{All} \cdot {v}_{Ney}}{\vert {v}_{Ney} \vert \vert {v}_{Ney} \vert} \qquad ( 9 )$$

where the numerator of the fraction contains the dot product of the two vectors, and the denominator of the fraction contains their magnitudes. The similarity score is simply the ratio of the scalar projection of v_{All} onto v_{Ney}, divided by the magnitude of v_{Ney}. Thus, if van is identical to the ideal Neyman allocation, S_{Ney} takes a value of 1. If v_{All} is not identical to the ideal Neyman allocation, S_{Ney} can have any non-negative value. (Without wishing to be bound by any particular theory, values of S_{Ney} cannot be negative because all allocations consist of non-negative coordinates, meaning that van and v_{Ney} both belong to the first quadrant of real space, and must form an angle θ between them of less than or equal to 90°. Since the dot product of v_{All} and v_{Ney} is equal to |v_{All}∥v_{Ney}|cos(θ), and since |v_{All}|, |v_{Ney}|, and cos(θ) are non-negative when θ is less than or equal to 90°, S_{Ney} is also non-negative, according to some embodiments.)

A Neyman allocation may have a similarity score (relative to an ideal Neyman allocation of the same size), S_{Ney}, of greater than or equal to 0.9, greater than or equal to 0.95, greater than or equal to 0.98, greater than or equal to 0.99, greater than or equal to 0.999, greater than or equal to 0.9999, or greater. In some embodiments, a Neyman allocation has a similarity score of less than or equal to 1.1, less than or equal to 1.05, less than or equal to 1.02, less than or equal to 1.01, less than or equal to 1.001, less than or equal to 1.0001, or less. Combinations of these ranges are also possible. For example, the Neyman allocation may have a similarity to an ideal Neyman allocation of the same size that is greater than or equal to 0.9 and less than or equal to 1.1, greater than or equal to 0.95 and less than or equal to 1.05, greater than or equal to 1.02 and less than or equal to 0.98, greater than or equal to 0.99 and less than or equal to 1.01, greater than or equal to 0.999 and less than or equal to 1.001, or greater than or equal to 0.9999 and less than or equal to 1.0001.

Once the number of measurement locations have been allocated to each stratum of a group of strata, the actual measurement locations may be determined by any of a variety of appropriate methods. In some embodiments, the measurement locations may be determined randomly within a stratum. For example, the measurement locations may be chosen uniformly randomly, according to some embodiments. Uniform randomness has, in the context of the present disclosure, been observed to present certain disadvantages for making robust estimates of bulk soil carbon content. For example, multiple measurement locations may be allocated to the same tile, even if variance within the tile is likely to be relatively limited. Thus, in some embodiments, measurement locations are determined such that each measurement location may be situated in separate tiles. The determination of the measurement locations such that no two measurement locations are situated within the same tile may be accomplished by any of a variety of appropriate methods (e.g., by selecting tiles randomly based on their relative size, and subsequently determining a measurement location randomly within the tile; or by determining measurement locations by a uniformly random sampling of the geographic region, and re-sampling any measurement locations that fall within a tile already sampled by another measurement location. Of course other appropriate sampling methods may also be employed as the disclosure is not so limited.

It has also been recognized, in the context of the present disclosure, that sampling near a boundary of a geographic region may reduce measurement accuracy. Accordingly, in some embodiments, measurement locations are not chosen within a buffer zone extending around an outer boundary of the geographic region. The buffer zone may have any of a variety of appropriate sizes, as discussed in greater detail with reference to the figures below.

As used herein, bulk soil carbon content may refer to a bulk soil carbon density (e.g., a molar- or mass-density of carbon within the soil) or to a total amount of carbon within a geographic region (e.g., a total mass or a total number of moles of carbon within the geographic region). Generally, an average bulk soil carbon density within a geographic region may be multiplied by a volume of the geographic region to obtain a total amount of carbon within the geographic region, and the use of the umbrella term “bulk soil carbon content” is therefore appropriate to describe either the measurement of bulk soil carbon density or total soil carbon. In other words, a method of determining bulk soil carbon content may determine bulk soil carbon density and/or total soil carbon within a geographic region, as the disclosure is not so limited.

One or more of the method steps provided herein may be performed using a computer, according to certain embodiments. Various components, such as processors and non-transitory computer readable memory discussed in greater detail below, may be used to perform various method steps. For example, a processor may be configured to perform a method. As another example, in some embodiments, a non-transitory computer readable storage medium may store processor executable instructions that, when executed by at least one processor, cause the at least one processor to perform any of the methods disclosed herein. These processes may either be done on site as part of an initial sampling of a geographic region and/or they may be performed offsite on a remotely located server or other computing device as the disclosure is not limited in this regards.

A sampling plan comprising a plurality of measurement locations may, in some embodiments, be outputted to a user. Additionally, or alternatively, a sampling plan may be stored in non-transitory computer readable storage memory, or may be exported from a computer, thus allowing the sampling plan to be recalled in future.

A sampling plan may be used to measure bulk soil carbon content within the geographic region, according to some embodiments. A method may comprise, at one or more measurement positions within the geographic region, measuring bulk soil carbon content. The bulk soil carbon content may be measured by any of a variety of appropriate methods. For example, the bulk soil carbon content may be measured in situ, e.g., using a soil penetrometer capable of determining bulk soil carbon content. As another example, the bulk soil carbon content may be determined by extracting a soil sample from a measurement location and measuring bulk soil carbon content elsewhere (e.g., in a laboratory environment, using a benchtop measurement system). Bulk soil carbon content may be determined, in some embodiments, as a function of depth, thereby allowing stratification of the geographic region into three-dimensional tiles, if desired. Alternatively, bulk soil carbon content may be determined as an average across the entire depth of the measurement location, which may be appropriate for two-dimensional stratifications of the geographic region.

It should be understood that while the various embodiments described herein are directed to determining a carbon content of soil, the various embodiments may be implemented to determine any appropriate soil property using an initial dataset either including that soil property and/or other soil properties related to the target soil property. Appropriate soil properties that may be sampled using the disclosed methods and systems may include, but are not limited to, bulk soil carbon content, soil organic carbon, bulk density, clay content, sand content, silt content, organic matter content, pH, cation exchange capacity (CEC), coarse fragment content, water content, nitrogen content, phosphorus content, potassium content, mineral-associated organic matter (MAOM), or particulate organic matter (POM).

In the various embodiments disclosed herein, the term uncertainty and other similar terms are used. However, it should be understood that any appropriate measurement of the uncertainty of a desired parameter within a region may be used in the different embodiments as the disclosure is not limited to any particular measure of uncertainty, this may include, but is not limited to, variance, standard deviation, standard error, margin of error, and/or any other appropriate measure of uncertainty.

Turning to the figures, specific non-limiting embodiments are described in further detail. It should be understood that the various systems, components, features, and methods described relative to these embodiments may be used either individually and/or in any desired combination as the disclosure is not limited to only the specific embodiments described herein.

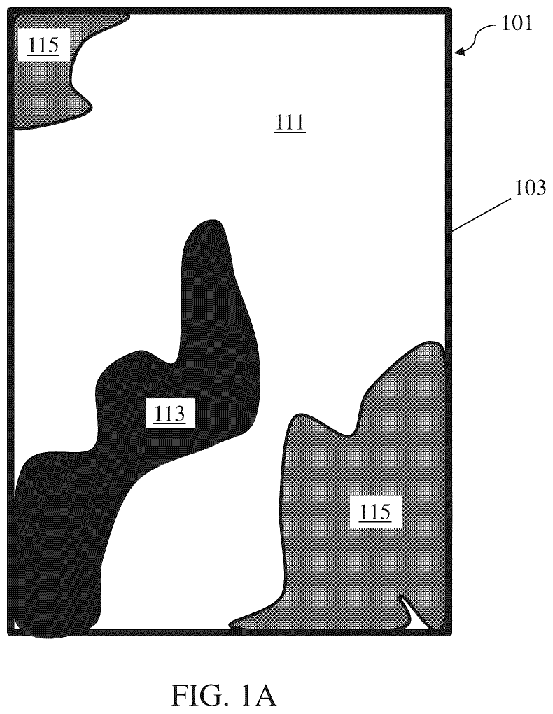

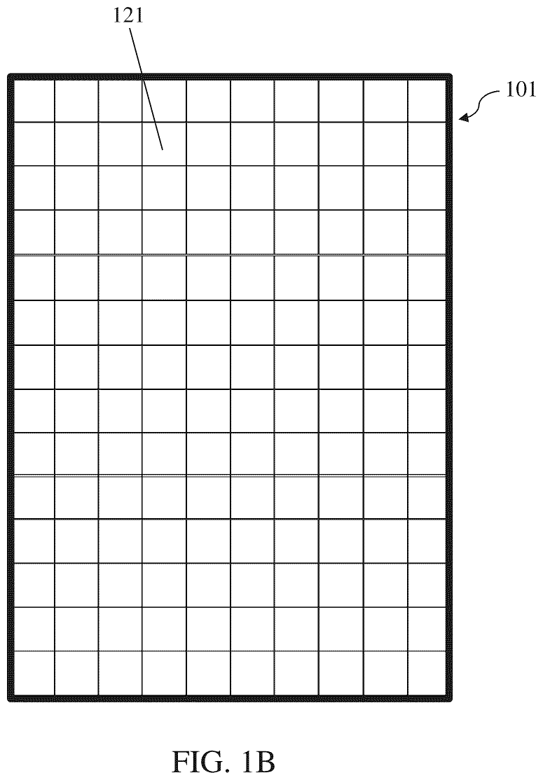

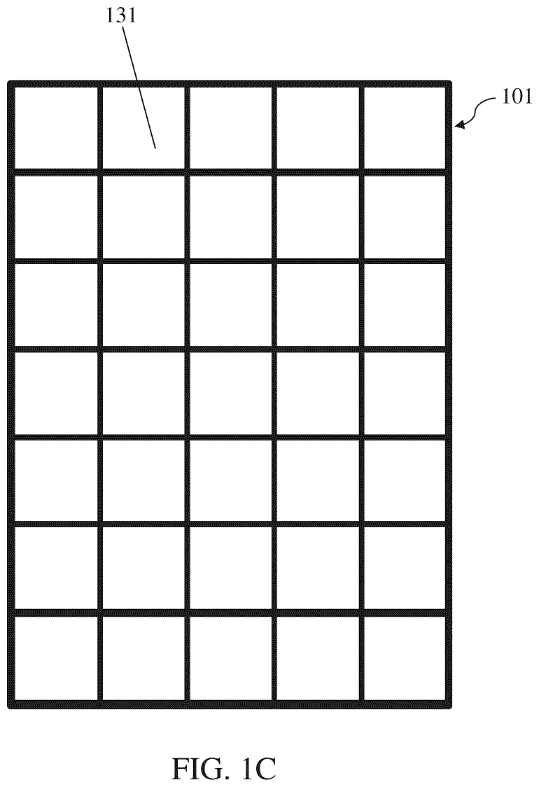

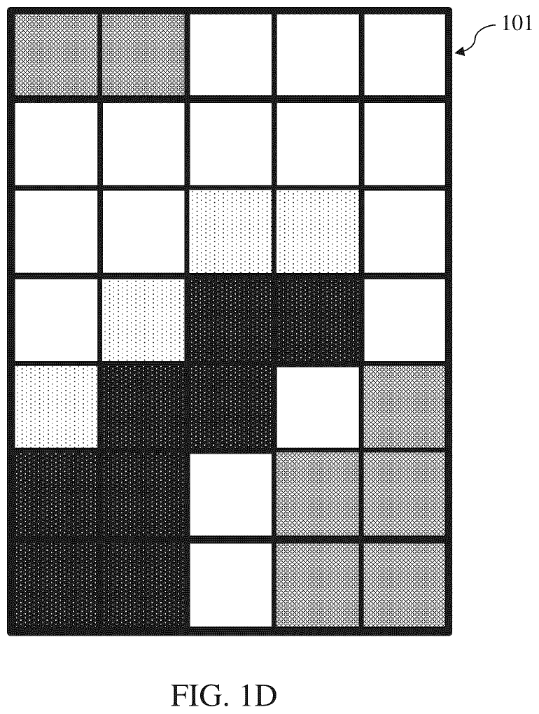

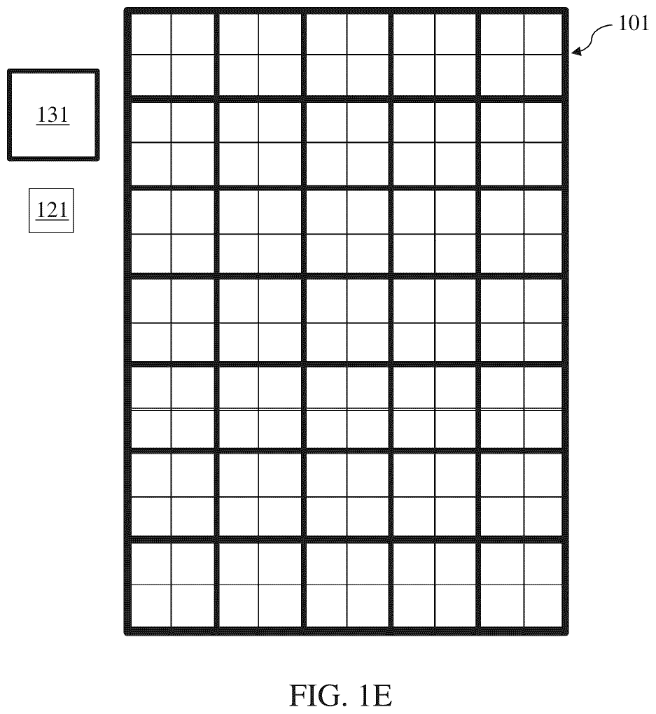

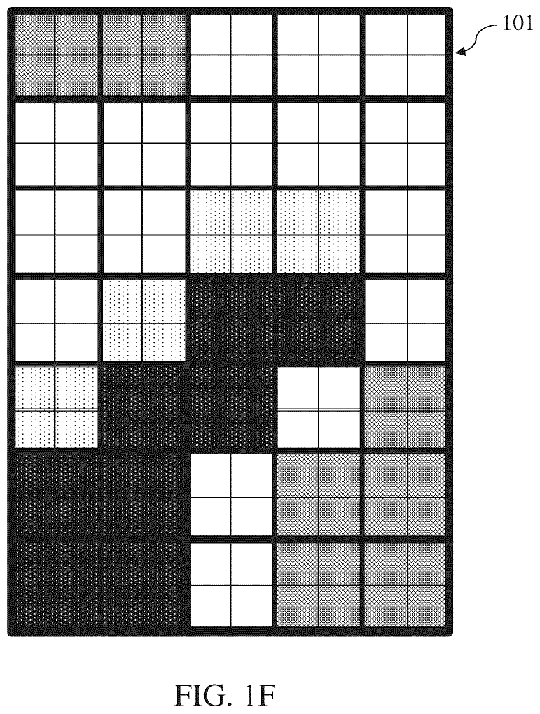

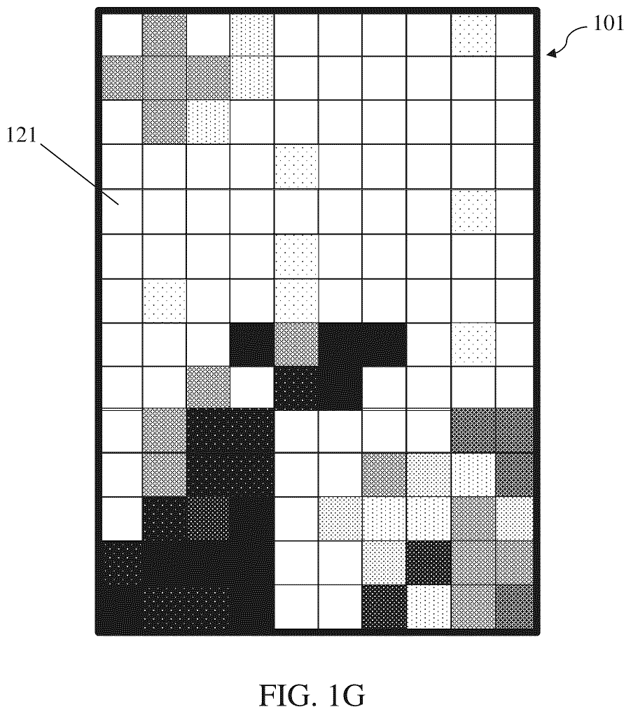

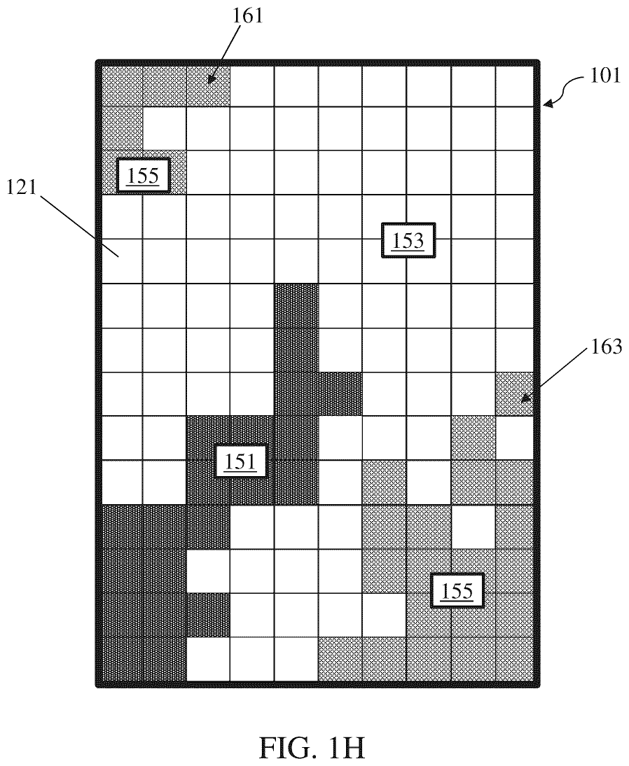

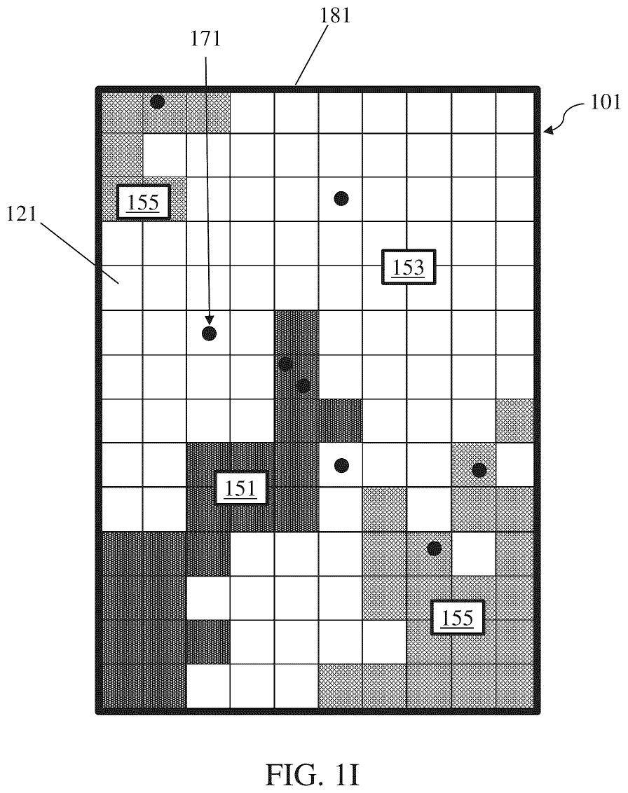

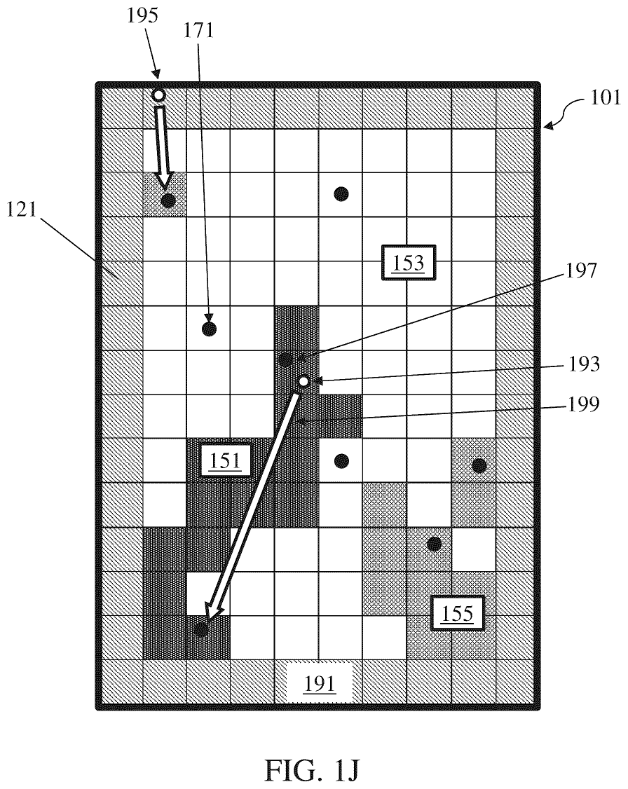

FIG. 1A presents an exemplary, schematic representation of a geographic region 101 bounded by boundary 103. Patterned regions 111, 113, and 115 represent portions of the geographic region with substantially different soil properties (schematically approximating a distribution of different soil types in the geographic region). Stratification of the geographic region to represent the different soil types may, in some embodiments, improve statistical approximation of bulk soil carbon content within geographic region 101.

Although geographic region 101 is presented as a rectangular region, it should of course be understood that the geographic region may have any geometry, as the disclosure is not limited to the geometry of any particular geographic region. The geographic region may have any of a variety of appropriate areas. In some embodiments, a geographic region has an area of greater than or equal to 0.1 km², greater than or equal to 0.5 km², greater than or equal to 1 km², greater than or equal to 2 km², greater than or equal to 5 km², greater than or equal to 10 km², or greater. In some embodiments, a geographic region has an area of less than or equal to 100 km², less than or equal to 80 km², less than or equal to 50 km², less than or equal to 20 km², less than or equal to 10 km², less than or equal to 5 km², or less. Combinations of these ranges are possible. For example, in some embodiments, a geographic region has an area of greater than or equal to 0.1 km² and less than or equal to 100 km². Other ranges, both higher and lower than those described above, are also possible, as the disclosure is not so limited.

The geographic region may have any of a variety of appropriate depths. In some embodiments, a geographic region has a depth of greater than or equal to 1 cm, greater than or equal to 5 cm, greater than or equal to 10 cm, greater than or equal to 20 cm, greater than or equal to 50 cm, greater than or equal to 100 cm, or greater. In some embodiments, a geographic region has a depth of less than or equal to 200 cm, less than or equal to 150 cm, less than or equal to 100 cm, less than or equal to 50 cm, less than or equal to 20 cm, less than or equal to 10 cm, or less. Combinations of these ranges are possible. For example, in some embodiments, a geographic region has a depth of greater than or equal to 1 cm and less than or equal to 200 cm. Other ranges are also possible, as the disclosure is not so limited.

Furthermore, although geographic region 101 is presented as a single, continuous geographic region, in some embodiments a geographic region may comprise a plurality of discontinuous regions, as the disclosure is not so limited. For example, a geographic region may comprise greater than or equal to 1, greater than or equal to 2, greater than or equal to 3, greater than or equal to 4, greater than or equal to 5, greater than or equal to 6, greater than or equal to 7, greater than or equal to 8, greater than or equal to 9, greater than or equal to 10, greater than or equal to 11, greater than or equal to 12, greater than or equal to 13, greater than or equal to 14, or more discontinuous regions. In some embodiments, a geographic region comprises less than or equal to 15, less than or equal to 14, less than or equal to 13, less than or equal to 12, less than or equal to 11, less than or equal to 10, less than or equal to 9, less than or equal to 8, less than or equal to 7, less than or equal to 6, less than or equal to 5, less than or equal to 4, less than or equal to 3, less than or equal to 2, or fewer discontinuous regions. Combinations of these ranges are possible. For example, in some embodiments, a geographic region comprises greater than or equal to 1 and less than or equal to 15 discontinuous regions. Other ranges are also possible.

Although FIG. 1A stratifies the soil uniformly into smooth strata, often, it is advantageous to subdivide the region into a plurality of tiles. A geographic region may be stratified two-dimensionally. For example, FIG. 1B presents geographic region 101, subdivided into two-dimensional tiles 121. As shown in FIG. 1B, tiles 121 may be rectangular tiles, and the geographic region is stratified two-dimensionally into rectangular tiles. The tiles may be regularly-shaped, in some embodiments. For example, the tiles may be regularly shaped rectangular tiles (i.e., “pixels”). However, the tiles may have any appropriate size and/or shape as the disclosure is not so limited. In some embodiments, the two-dimensional tiles of a plurality of tiles have an average maximum transverse dimension of greater than or equal to 1 m, 2 m, 3 m, 4 m, 5 m, 10 m, 15 m, or greater. In some embodiments, the two-dimensional tiles have an average maximum transverse dimension of less than or equal to 20 m, 15 m, 10 m, 5 m, 4, m, 3 m, or less. Combinations of these ranges are possible. For example, in some embodiments, the two-dimensional tiles have an average maximum transverse dimension of between or equal to 1 m and 20 m, 10 m and 20 m, and/or any other appropriate dimension. Other ranges, both greater and less than those described above, are also possible, as the disclosure is not so limited.

In some embodiments, the two-dimensional tiles of a plurality of tiles may also have an average minimum transverse dimension of greater than or equal to 1 m, 2 m, 3 m, 4 m, 5 m, 10 m, 15 m, or greater. In some embodiments, the two-dimensional tiles have an average minimum transverse dimension of less than or equal to 20 m, 15 m, 10 m, 5 m, 4, m, 3 m, or less. Combinations of these ranges are possible. For example, in some embodiments, the two-dimensional tiles have an average minimum transverse dimension of between or equal to 1 m and 20 m, 10 m and 20 m, and/or any other appropriate dimension. Other ranges, both greater and less than those described above, are also possible, as the disclosure is not so limited.

In some embodiments, two-dimensional tiles of a plurality of tiles have an average area of greater than or equal to 1 m², greater than or equal to 10 m², greater than or equal to 50 m², greater than or equal to 100 m², greater than or equal to 200 m², greater than or equal to 300 m², or greater. In some embodiments, the two-dimensional tiles have an average area of less than or equal to 500 m², less than or equal to 400 m², less than or equal to 300 m², less than or equal to 200 m², less than or equal to 100 m², less than or equal to 50 m², less than or equal to 10 m², or less. Combinations of these ranges are possible. For example, in some embodiments, the two-dimensional tiles have an average area of between or equal to 1 m² and 500 m², 50 m² and 200 m², and/or any other appropriate combination. Other ranges, both greater and less than those described above, are also possible, as the disclosure is not so limited.

Geographic region 101 is, for the purposes of illustration, represented as a two-dimensional geographic area—however, it should be understood that the geographic region has a depth in an out-of-plane direction of FIG. 1A. In some embodiments, a geographic region such as geographic region 101 may be stratified three-dimensionally. The geographic region may be stratified into rectangular prisms. In some embodiments, the geographic region may be stratified into regular volumetric tiles. For example, the geographic region may be stratified into regular rectangular prisms (i.e., voxels). The three-dimensional tiles may have any of a variety of appropriate sizes and/or shapes. For example, the three dimensional areas may have any of the above mentioned transverse dimensions, aspect ratios, and/or areas in the width and breadth dimension of the geographic region, coupled with an appropriate depth. The tiles may have any of a variety of appropriate depths. In some embodiments, three-dimensional tiles of a plurality of tiles have an average depth of greater than or equal to 1 cm, greater than or equal to 5 cm, greater than or equal to 10 cm, greater than or equal to 20 cm, greater than or equal to 50 cm, greater than or equal to 100 cm, or greater. In some embodiments, the three dimensional tiles have an average depth of less than or equal to 200 cm, less than or equal to 150 cm, less than or equal to 100 cm, less than or equal to 50 cm, less than or equal to 20 cm, less than or equal to 10 cm, or less. Combinations of these ranges are possible. For example, in some embodiments, the three dimensional tiles have an average depth of greater than or equal to 1 cm and less than or equal to 200 cm. Other ranges are also possible, as the disclosure is not so limited.

As discussed above, in some embodiments, a method comprises obtaining an initial dataset, and the initial dataset may comprise a plurality of tiles subdividing the geographic region. For example FIG. 1C presents a schematic representation of a plurality of tiles 131 subdividing geographic region 101 that are associated with an initial dataset. The initial dataset may include one or more properties associated with a plurality of tiles. Any of a variety of appropriate tilings may be associated with the initial dataset, including any of the tilings described above in the context of subdividing geographic region 101.

The tiles of the initial dataset may be associated with one or more soil properties. For example, FIG. 1D shows tiles 131 of FIG. 1C, with a shading indicating a value of a soil property associated with the tile and obtained as part of the initial dataset. The plurality of tiles associated with the initial dataset may differ from the plurality of tiles used for stratification. For example, FIG. 1E overlays tiles 121 of FIG. 1B with tiles 131 of FIG. 1D, illustrating the differences between the subdivisions of geographic region 101 by each plurality of tiles. In some embodiments, a method comprises mapping a second plurality of tiles obtained from an initial dataset into a first plurality of tiles used for stratification. For example, in some embodiments, tiles 121 used for stratification are subdivisions of tiles 131 from the initial dataset, as shown in FIG. 1E, where each tile 131 is subdivided into four tiles 121. In some embodiments, tiles of an initial dataset may be subdivided into greater than or equal to 2, 3, 4, 5, 10, 20, 30, or other appropriate number of subdivided tiles. The initial tiles may also be subdivided into less than or equal to 50, 40, 30, 20, 10, or any other appropriate number of tiles used for stratification. For example, the initial tiles may be subdivided into 2 to 50 sub-tiles, 20 to 40 sub-tiles, approximately 30 sub-tiles and/or any other appropriate combination of the forgoing or other appropriate number of tiles. Of course, embodiments where the first plurality of tiles 121 and the second plurality of tiles 131 do not match perfectly, or where the same tiles from the initial dataset are used for stratification are also possible, as the disclosure is not so limited.

Soil properties of tiles 121 used for stratification of geographic region 101 may be determined from the initial dataset in any of a variety of appropriate ways. For example, tiles 121 may take soil properties directly from tiles of the initial dataset as shown in FIG. 1F. As shown, tiles 121 of FIG. 1F have soil properties directly taken from the soil properties of tiles 131 shown in FIG. 1D.

Alternatively, in some embodiments, soil properties of tiles 121 used for stratification may be obtained by randomly varying soil properties based on a statistical distribution of the soil properties within the initial dataset. For example, the initial dataset may include average values and variances of one or more soil properties, and rather than simply applying the average values of tiles 131 to tiles 121, as was done in FIG. 1F, the values of the soil properties at tiles 121 may be determined by determining the values, at random, from a statistical distribution associated with the average values and variances of the one or more soil properties provided in the initial dataset. An example is shown in FIG. 1G, wherein tiles 121 have randomly varying shading predicted by the soil properties of initial tiles 131 (as shown in FIG. 1D). Introducing random variation into the values used for stratification may, in some cases, increase uncertainty in the model, advantageously increasing the likelihood that a sampling plan can accurately sample the geographic region.

Once the soil property or properties have been assigned to tiles 121, the tiles may be stratified. FIG. 1H provides an exemplary, schematic illustration of the stratification of geographic region 101 into three strata, stratum 151, stratum 153, and stratum 155. The strata formed by clustering may have any appropriate spatial distribution within the geographic region. For example, strata 151 and 153 span continuous portions of the geographic region. According to some embodiments, the strata include discontinuous tiles or groups of tiles. For example, stratum 155 may be discontinuous, because it includes a first tile 161 and a second tile 163 that have been clustered into the same stratum as a result of their similar soil properties, but that are geographically isolated from one another within geographic region 101, such that there is no path between them passing exclusively through stratum 155, to which they both belong. Discontinuous strata may be common when stratifying geographic regions. For example, in some embodiments a geographic region is a rectangular field that is irrigated using a circular pivot irrigation system, with the result that a central circle in the field is better irrigated than the field's corners. In some such embodiments, the soil in the central circle is stratified into a different stratum than the soil at the corners of the field which may either be clustered together or separately from one another depending on the embodiment.

FIG. 1I provides a non-limiting, schematic illustration of geographic region 101 comprising strata 151, 153, and 155 as shown in FIG. 1H, and shows a plurality of measurement locations 171 that have been determined using the strata. Measurement locations 171 may be sampled with random uniformity, as they were in FIG. 1I.

However, as discussed briefly above, in some embodiments, the measurement locations are excluded from a buffer zone extending around outer boundary 181 of geographic region 101, and in some embodiments, multiple measurement locations 171 may not be situated within a same tile. FIG. 1J therefore schematically illustrates the same geographic region 101, strata 151, 153, and 155, and measurement locations 171 of FIG. 1I, this time corrected to exclude sampling of the buffer zone 191 (indicated by black tiles) and to exclude repeated sampling from a same tile. Measurement locations 193 and 195, represented by white circles, correspond to measurement locations of FIG. 1I that disobeyed these constraints by either falling within buffer zone 191 (measurement location 193 falls within buffer zone 191) or by being sampled within a same tile 121 as another measurement location (measurement location 195 falls within a same tile as measurement location 197). Accordingly, these points have been re-sampled, as indicated by white arrows 199. It should be noted that a buffer zone 191 only extends from an area boundary of a geographic region (e.g., area boundary 181 of FIG. 1I), and includes the entire depth of the geographic region. No buffer zone extends from a major surface (a surface parallel to both the width and breadth dimensions) of the geographic region, in most embodiments.

The buffer zone may have any of a variety of appropriate widths, depending on the embodiment. In some embodiments, a buffer zone has a width of greater than or equal to 0.5 m, greater than or equal to 1 m, greater than or equal to 2 m, greater than or equal to 4 m, greater than or equal to 6 m, greater than or equal to 8 m, or greater. In some embodiments, a buffer zone has a width of less than or equal to 14 m, less than or equal to 12 m, less than or equal to 10 m, less than or equal to 8 m, less than or equal to 6 m, less than or equal to 4 m, or less. Combinations of these ranges are possible. For example, in some embodiments, a buffer zone has a width of greater than or equal to 0.5 m and less than or equal to 14 m. Other ranges, both greater and less than those described above, are also possible, as the disclosure is not so limited.

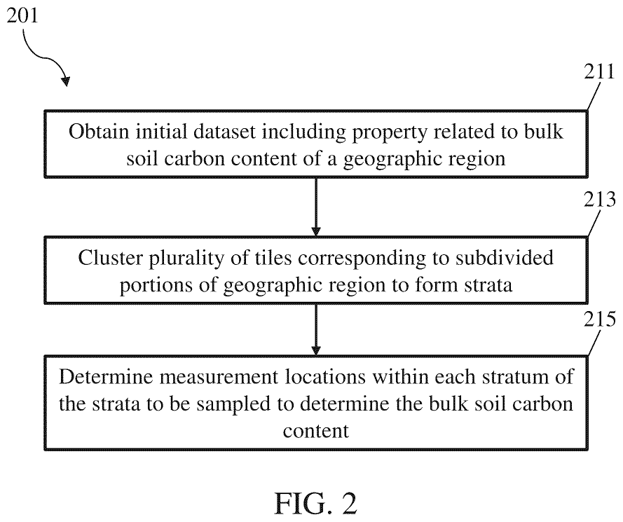

FIG. 2 presents a schematic illustration of method 201 for determining measurement locations, according to some embodiments. Method 201 comprises step 211 of obtaining an initial dataset including a property related to bulk soil carbon content of a geographic region. As discussed above, the initial dataset may comprise data regarding one or more exemplary soil properties related to bulk soil carbon content, such as bulk density, moisture content, clay content, and organic matter content. According to some embodiment, the initial dataset provides geographical information regarding the one or more soil properties, e.g., by associating them with a plurality of tiles representing a geographic region, as discussed above with reference to FIG. 1F. Of course, embodiments where the initial dataset does not provide geographical information about the soil properties within the geographic region are also envisioned, as the disclosure is not so limited.

It should, of course, be noted that more than one initial dataset may be obtained, and that in some embodiments a plurality of datasets may be obtained during the performance of a method. For example, a first dataset comprising a modelled distribution of soil properties and a second dataset comprising a plurality of preliminary measurements of one or more soil properties can both be used, in some embodiments. As another example, predicted soil properties from a plurality of models may be used. The use of multiple datasets may, in some embodiments, improve the initial accuracy of stratification and variance estimation in subsequent method steps, which may be advantageous for developing good soil plans, according to some embodiments.

As shown in FIG. 2, method 201 may further comprise a step of clustering a plurality of tiles corresponding to subdivided portions of the geographic region, in order to form strata. Any of a variety of appropriate clustering algorithms may be used. For example, the clustering algorithm may be a centroid-based clustering (e.g., k-means clustering) algorithm, a k-medians clustering algorithm, a density-based clustering algorithm, a distribution-based clustering algorithm, or any of a variety of other suitable clustering algorithms, including but not limited to algorithms implemented in Birch, MeanShift, DBSCAN, Affinity Propagation, OPTICS, and/or Gaussian Mixture Model Clustering. Any suitable number of tiles may be provided to the clustering algorithm (e.g., greater than 50, greater than 100, greater than 500, etc.). The result of the clustering algorithm is to group each tile into one of the strata.

The clustering algorithm may be used to cluster the tiles into any of a variety of appropriate numbers of strata based on an obtained desired number of strata. In some embodiments, a clustering algorithm is used to cluster tiles into greater than or equal to 1, greater than or equal to 2, greater than or equal to 3, greater than or equal to 4, greater than or equal to 5, greater than or equal to 8, greater than or equal to 10, greater than or equal to 12, greater than or equal to 15, greater than or equal to 20, greater than or equal to 30, greater than or equal to 40, or more strata. In some embodiments, a clustering algorithm is used to cluster tiles into less than or equal to 50, less than or equal to 40, less than or equal to 30, less than or equal to 20, less than or equal to 15, less than or equal to 12, less than or equal to 10, less than or equal to 8, less than or equal to 5, less than or equal to 3, less than or equal to 2, or fewer strata. Combinations of these ranges are possible. For example, in some embodiments, a clustering algorithm is used to cluster tiles into greater than or equal to 1 and less than or equal to 50 strata. Other ranges are also possible, as the disclosure is not so limited.

The clustering may be performed on the basis of a single soil property (e.g., bulk soil carbon content, bulk density, moisture content, organic matter content, clay content) associated with the plurality of tiles. However, clustering may, in some embodiments, be performed using a plurality of soil properties obtained from the initial dataset. For example, clustering may be performed based on greater than or equal to 1, greater than or equal to 2, greater than or equal to 3, or more distinct soil properties. In some embodiments, clustering is performed on up to 10, up to 5, or up to 3 distinct soil properties. Combinations of these ranges are also possible. For example, clustering may be performed on greater than or equal to 1 and up to 10 distinct soil properties. It may be advantageous to perform clustering using exactly one soil property, e.g., since this may be all that is needed to accurately stratify the geographic region. However, clustering using a plurality of soil properties may be advantageous for differentiating between soil types that differ, but that coincidentally share similar values of a particular soil property. For instance, a first soil type and a second soil type may have a similar moisture, but different clay and organic matter contents, such that the first soil type is organic-rich and the second soil type is clay-rich. Using multiple soil properties to stratify the geographic region may help to distinguish the first soil type from the second soil type.

In a third step 215, method 201 comprises determining measurement locations within each stratum of the group of strata to be sampled in order to determine the bulk soil carbon content of the geographic region. The measurement locations may be determined by any of a variety of appropriate methods, including uniformly random sampling of locations based on a Neyman allocation within the geographic region, as discussed above. It should, of course, be understood that the method does not necessarily require the actual determination of bulk soil carbon content. In some embodiments, the measurement locations are determined in order to determine the bulk soil carbon content of the geographic region, but merely for the purpose of developing a sampling plan, which need not actually be executed. Alternatively, of course, the bulk soil carbon content can actually be determined using a sample plan comprising the determined measurement locations.

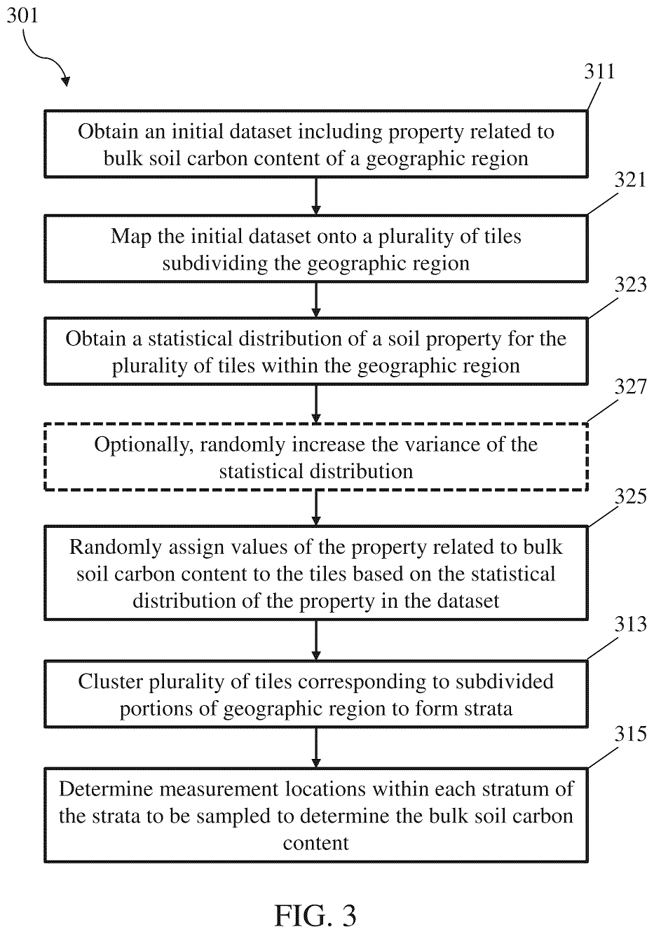

FIG. 3 presents a schematic illustration of another method 301, according to some embodiments. Method 301 comprises steps 311, 313, and 315, corresponding to method steps 211, 213, and 215 respectively, as discussed with reference to FIG. 2 above. However, method 301 further comprises steps 321, 323, 325, and optional step 327 (indicated by the dashed line) as described in greater detail below. Step 321 comprises mapping the initial dataset onto a plurality of tiles subdividing the geographic region. This may be accomplished in any of a variety of ways. For example, the plurality of tiles may be a first plurality of tiles, which may be identical to or may subdivide a second plurality of tiles associated with the initial dataset.

As another example, a first plurality of tiles may be mismatched with a second plurality of tiles of the initial dataset, and a method may comprise assigning values of soil properties to the tiles of the first plurality of tiles by identifying tiles of the second plurality that overlap each tile of the first plurality, and optionally by identifying a relative weight that each overlapping tile of the second plurality contributes towards the value of the soil properties of the tiles of the first plurality. Such weights may be determined by any of a variety of appropriate methods. For example, weights may be assigned based on overlap area or overlap volume.

Values of the soil properties of the first plurality of tiles subdividing the geographic region may be determined by any of a variety of appropriate methods. In method 301, for example, the values of the soil properties of the tiles are determined by obtaining a statistical distribution of a soil property for the plurality of tiles within the geographic region (step 323) and randomly assigning values of the soil property to the tiles of the first plurality based on the statistical distribution of the soil property (step 325). Any of a variety of appropriate statistical distributions may be used, depending on the embodiment. For example, in some embodiments, the statistical distribution is a Gaussian function (e.g., a univariate Gaussian representing the distribution of a single soil property, or a multivariate Gaussian representing the distributions of a plurality of soil properties). The statistical distribution may be characterized by a statistical average and a statistical variance provided in the initial dataset. Of course, other appropriate statistical distributions and measurements of variability may also be used. In some embodiments, different tiles of a plurality of tiles of the initial dataset are associated with different statistical distributions. Values of the one or more soil properties associated with the first plurality of tiles can, in some embodiments, be obtained by sampling one or more statistical distributions of the initial dataset, weighted by the mapping of the initial dataset onto the first plurality of tiles. For example, a tile used for the stratification analysis, which falls totally within a tile of the initial dataset, may be assigned one or more soil properties based on a statistical distribution of soil properties associated with the tile of the initial dataset. As another example, a tile used for the stratification analysis, which overlaps multiple tiles of the initial dataset, may be assigned one or more soil properties based on a weighted combination of the statistical distribution of soil properties associated with the tiles of the initial dataset. It should, of course, be understood that the disclosure is not limited to any particular method of obtaining statistical distributions or assigning values of soil properties to tiles used for stratification, as any of a variety of other approaches may be suitable.

In some instances, a variance associated with a portion of an initial dataset may be artificially low such that it does not reflect an actual variance of the soil property. For example, this may be an artifact of the modeling used to produce the initial dataset or as an artifact of a limited sampling used to estimate the variance. A low variance in an initial dataset, if undesirably low, may cause a model to underestimate the variance expected from measuring bulk soil carbon content at a plurality of measurement locations within the geographic region. Underestimating a variance associated with a plurality of measurement locations could result in underestimation of a minimum number of measurement locations needed to measure bulk soil carbon content with a target uncertainty, or in an expected minimum uncertainty in bulk soil carbon content that is incorrectly low if the soil is sampled with a target number of measurement locations. Accordingly, in instances in which the variance of a tile is less than a threshold variance, or other measure of minimum desirable uncertainty, the variance of the tile may be artificially increased, as indicated by optional step 327 of method 301. The variance of one or more of the statistical distributions may be artificially increased based on human judgement, may be artificially increased according to an objective criterion, such as an inherent minimum variance associated with a model of the initial dataset, and/or may be set to an expected minimum variance associated with the soil property being estimated. Increasing the variance may, in some embodiments, advantageously, make the model more conservative. In some embodiments, a method comprises increasing the variance of some, but not all, of the tiles. Of course, in some embodiments, no tiles have an artificially increased variance, and in some embodiments, all of the tiles may have an artificially increased variance, as the disclosure is not so limited.

The variance of the statistical distribution of a soil property associated with the initial dataset may generally be increased by any of a variety of appropriate amounts. In some embodiments, a variance of a statistical distribution is increased by a factor of greater than or equal to 1, greater than or equal to 1.2, greater than or equal to 1.5, greater than or equal to 2, greater than or equal to 3, greater than or equal to 5, or greater. In some embodiments, a variance of a statistical distribution is increased by a factor of less than or equal to 10, less than or equal to 8, less than or equal to 5, or less. Combinations of these ranges are possible. For example, in some embodiments, a variance of a statistical distribution is increased by a factor of greater than or equal to 1 and less than or equal to 10. Other ranges, both higher and lower than those described above, are also possible, as the disclosure is not so limited. Of course, embodiments, in which the variance is simply set to a minimum expected variance value are also contemplated as the disclosure is not limited to how a variance associated with an initial dataset and/or the tiles formed using the dataset is determined.

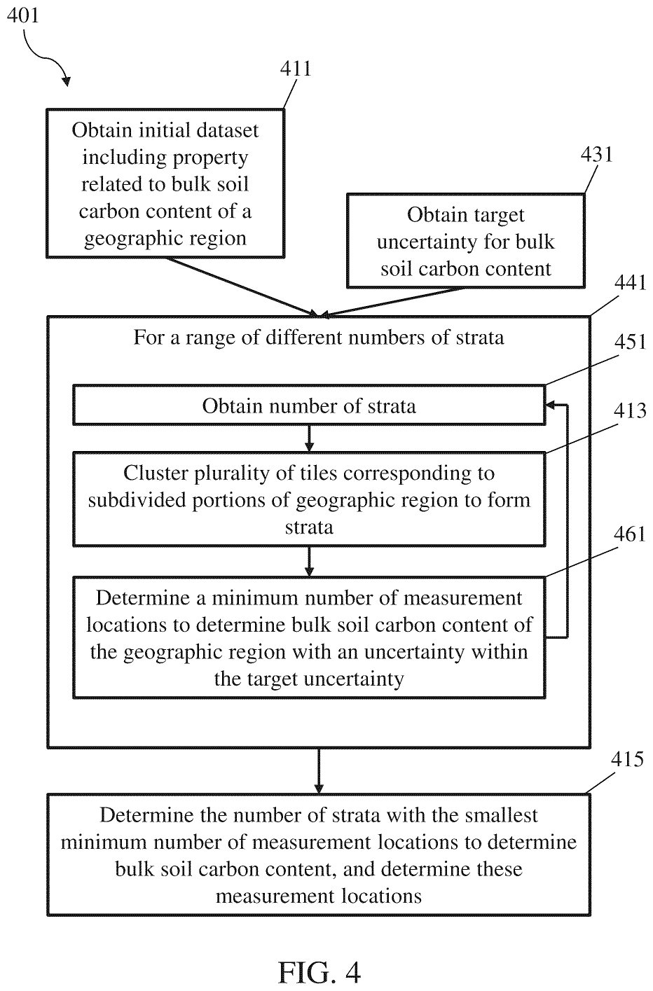

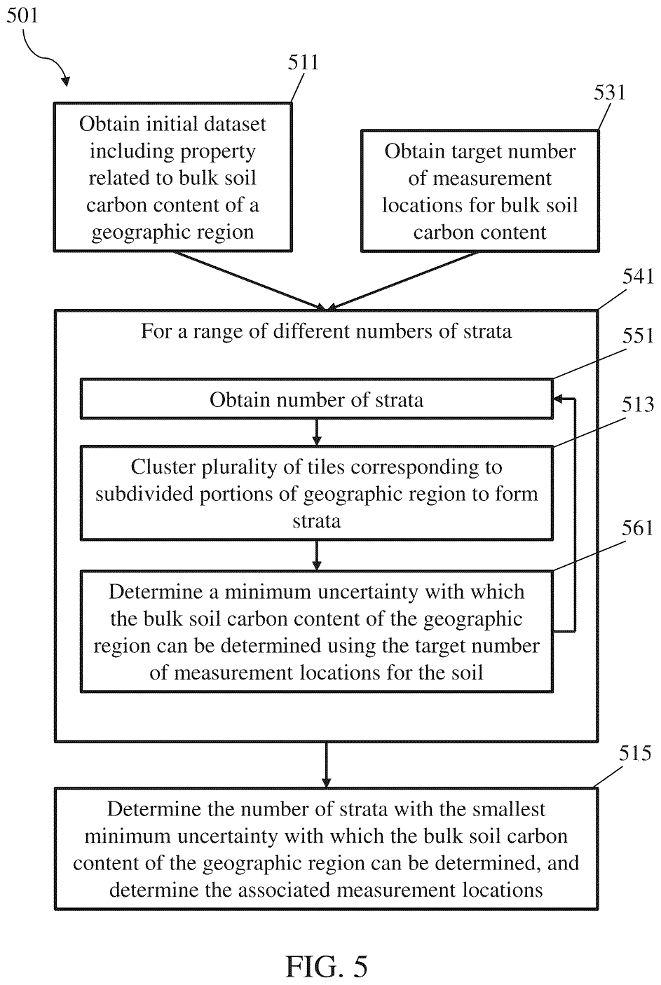

As discussed above, in some embodiments, a clustering step may be iterated in order to identify a number of strata that minimizes a number of measurement locations required to accurately sample bulk soil carbon content in the geographic region within a target uncertainty, or that minimizes an expected uncertainty for a target number of measurement locations. These concepts are represented, schematically, in FIGS. 4 and 5, respectively.

FIG. 4 schematically represents a method 401, comprising step 411 of obtaining an initial dataset (which is analogous to steps 211 and 311 of FIGS. 2 and 3, respectively). Method 401 further comprises a step 431 of obtaining a target uncertainty for bulk soil carbon content. As discussed above, the target uncertainty for bulk soil carbon content can be user provided or can take a default value—and this step can be performed before, after, or concurrently with step 411. In some embodiments, method 401 comprises an iterative process 441, comprising iteration of steps 451 of obtaining a number of strata, 413 of clustering tiles corresponding to subdivided portions of the geographic region to form strata, and 461 of determining a minimum number of measurement locations required to determine the bulk soil carbon content of the geographic region with an uncertainty below the target uncertainty. Step 451 of obtaining a number of strata may be performed by of a variety of appropriate methods. For example, the number of strata may be iterated systematically through a range of possible numbers of strata (e.g., from 1 stratum to N strata, where N is a maximum permitted number of strata, or from K strata to N strata, where K is a minimum permitted number of strata). In some embodiments, non-sequential numbers of strata may be obtained.

Step 413 of clustering the tiles into the number of strata obtained is similar to steps 213 of method 201 and 313 of method 301, as described with reference to the figures above.

Step 461 of determining a minimum number of measurement locations to determine a bulk soil carbon content of the geographic region with an uncertainty within a target uncertainty may be performed according to any appropriate method. For example, step 461 may be performed by iteratively allocating an increasing number of possible measurement locations to the strata (e.g., using a Neyman allocation) and predicting the resulting uncertainty (e.g., variance, standard error), repeating the iteration until the allocation of measurement locations is expected to have uncertainty within the target uncertainty. In some embodiments, the method comprises one or more additional steps of adjusting the Neyman allocations of possible measurement locations, in order to determine whether expected uncertainty can be decreased relative to the uncertainty associated with the Neyman allocation. Other embodiments are also possible, as the disclosure is not so limited.

Steps 451, 413, and 416 may be performed consecutively, and may be iterated any for any of a variety of appropriate numbers of strata (e.g., for any number of strata into which tiles may be clustered, for which exemplary ranges are provided above). The minimum number of measurement locations associated with each number of strata may be stored for later use.

Once iterative process 441 is complete, method 401 comprises step 415 of determining the number of strata with the smallest minimum number of measurement locations associated with stratification into any of the numbers of strata identified in process 441, and determining these measurement locations. The smallest minimum number of measurement locations may be determined by any of a variety of appropriate methods. For example, the smallest minimum number of measurement locations may be determined by comparing the number of measurement locations associated with the different strata groups stored during process 441. The measurement locations themselves may be determined as discussed above with reference to step 215 of method 201.

FIG. 5 presents method 501, which is similar to method 401 of FIG. 4, but which minimizes a target number of measurement locations for bulk soil carbon content, rather than minimizing uncertainty for a given target number of measurement locations. Accordingly, method 501 comprises steps 511, 551, and 513, which are identical to steps 411, 451 and 453 of method 401 as discussed above. Method 501 further comprises a step 531 of obtaining a target number of measurement locations for bulk soil carbon content. Steps 551 and 513 are part of iterative process 541, which further comprises step 561 of determining a minimum uncertainty (e.g., variance, standard error) with which the bulk soil carbon content of the geographic region can be measured using the target number of measurement locations for the soil. Step 561 may be performed using any of a variety of appropriate methods. In particular, in some embodiments the minimum uncertainty may be determined by preparing one or more allocations of measurement locations between the strata, and estimating the uncertainty. In some embodiments, the allocation that minimizes uncertainty is a Neyman allocation, as is described in greater detail above.

Finally, method 501 comprises step 515, which is similar to step 415 of method 401, except that instead of determining the minimum number measurement locations, the method comprises determining the smallest minimum uncertainty that can be obtained for any of the numbers of strata obtained during process 541 by comparing the uncertainty for the different strata groups to determine the strata group and sampling plan that provides the minimum uncertainty for the target number of measurement locations. The measurement locations themselves may be determined as discussed above with reference to step 215 of method 201.

The above-described embodiments of the technology described herein can be implemented in any of numerous ways. For example, the embodiments may be implemented using hardware, software or a combination thereof. When implemented in software, the software code can be executed on any suitable processor or collection of processors, whether provided in a single computing device or distributed among multiple computing devices. Such processors may be implemented as integrated circuits, with one or more processors in an integrated circuit component, including commercially available integrated circuit components known in the art by names such as CPU chips, GPU chips, microprocessor, microcontroller, or co-processor. Alternatively, a processor may be implemented in custom circuitry, such as an ASIC, or semicustom circuitry resulting from configuring a programmable logic device. As yet a further alternative, a processor may be a portion of a larger circuit or semiconductor device, whether commercially available, semi-custom or custom. As a specific example, some commercially available microprocessors have multiple cores such that one or a subset of those cores may constitute a processor. Though, a processor may be implemented using circuitry in any suitable format.

Further, it should be appreciated that a computing device may be embodied in any of a number of forms, such as a rack-mounted computer, a desktop computer, a laptop computer, or a tablet computer. Additionally, a computing device may be embedded in a device not generally regarded as a computing device but with suitable processing capabilities, including a Personal Digital Assistant (PDA), a smart phone, tablet, or any other suitable portable or fixed electronic device.

Also, a computing device may have one or more input and output devices. These devices can be used, among other things, to present a user interface. Examples of output devices that can be used to provide a user interface include display screens for visual presentation of output and speakers or other sound generating devices for audible presentation of output. Examples of input devices that can be used for a user interface include keyboards, individual buttons, and pointing devices, such as mice, touch pads, and digitizing tablets. As another example, a computing device may receive input information through speech recognition or in other audible format.

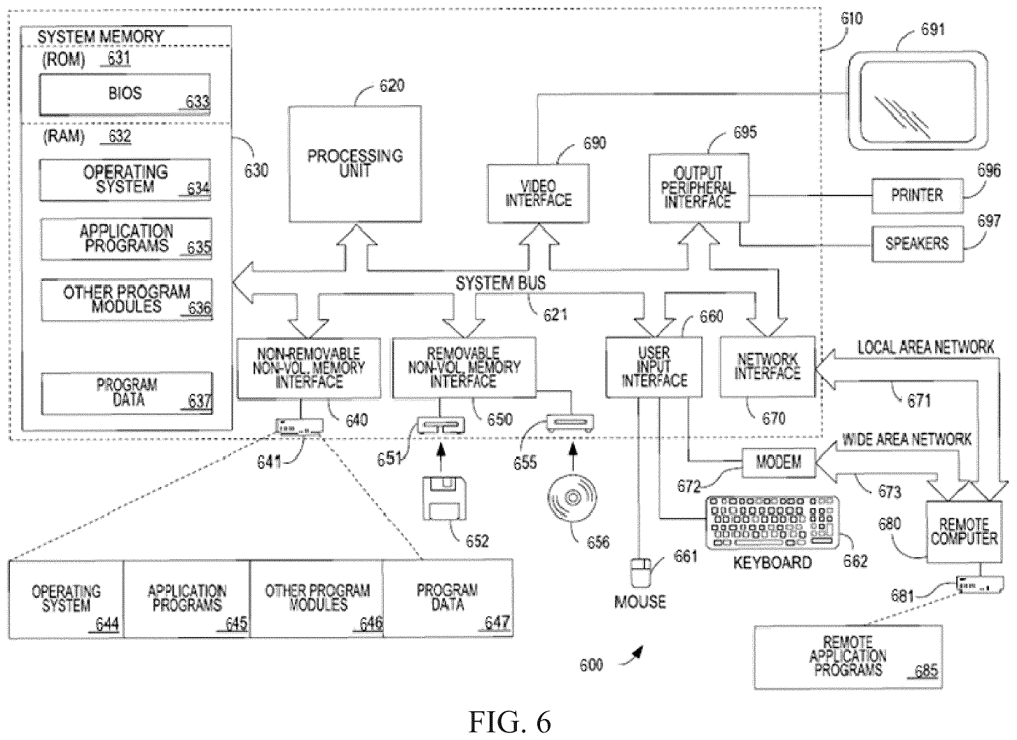

With reference to FIG. 6, an exemplary system for implementing aspects of the disclosure includes a general purpose computing device in the form of a computer 610 or other appropriate computing device. For example, the depicted computing device may be used as a soil measurement planning system configured to implement any of the methods disclosed herein. Components of computer 610 may include, but are not limited to, a processing unit 620, a system memory 630, and a system bus 621 that couples various system components including the system memory to the processing unit 620. The system bus 621 may be any of several types of bus structures including a memory bus or memory controller, a peripheral bus, and a local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus also known as Mezzanine bus.

Computer 610 typically includes a variety of computer readable media. Computer readable media can be any available media that can be accessed by computer 610 and includes both volatile and nonvolatile media, removable and non-removable media. By way of example, and not limitation, computer readable media may comprise computer storage media and communication media. Computer storage media includes both volatile and nonvolatile, removable and non-removable media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can accessed by computer 610. Communication media typically embodies computer readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of the any of the above should also be included within the scope of computer readable media.

The system memory 630 includes computer storage media in the form of volatile and/or nonvolatile memory such as read only memory (ROM) 631 and random access memory (RAM) 632. A basic input/output system 633 (BIOS), containing the basic routines that help to transfer information between elements within computer 610, such as during start-up, is typically stored in ROM 631. RAM 632 typically contains data and/or program modules that are immediately accessible to and/or presently being operated on by processing unit 620. By way of example, and not limitation, FIG. 10 illustrates operating system 634, application programs 635, other program modules 636, and program data 637.

The computer 610 may also include other removable/non-removable, volatile/nonvolatile computer storage media. By way of example only, FIG. 10 illustrates a hard disk drive 641 that reads from or writes to non-removable, nonvolatile magnetic media, a magnetic disk drive 651 that reads from or writes to a removable, nonvolatile magnetic disk 652, and an optical disk drive 655 that reads from or writes to a removable, nonvolatile optical disk 656 such as a CD ROM or other optical media. Other removable/non-removable, volatile/nonvolatile computer storage media that can be used in the exemplary operating environment include, but are not limited to, magnetic tape cassettes, flash memory cards, digital versatile disks, digital video tape, solid state RAM, solid state ROM, and the like. The hard disk drive 641 is typically connected to the system bus 621 through an non-removable memory interface such as interface 640, and magnetic disk drive 651 and optical disk drive 655 are typically connected to the system bus 621 by a removable memory interface, such as interface 650.

The drives and their associated computer storage media discussed above and illustrated in FIG. 10, provide storage of computer readable instructions, data structures, program modules and other data for the computer 610. In FIG. 10, for example, hard disk drive 641 is illustrated as storing operating system 644, application programs 645, other program modules 646, and program data 647. Note that these components can either be the same as or different from operating system 634, application programs 635, other program modules 636, and program data 637. Operating system 644, application programs 645, other program modules 646, and program data 647 are given different numbers here to illustrate that, at a minimum, they are different copies. A user may enter commands and information into the computer 610 through input devices such as a keyboard 662 and pointing device 661, commonly referred to as a mouse, trackball or touch pad. Other input devices (not shown) may include a microphone, joystick, game pad, satellite dish, scanner, or the like. These and other input devices are often connected to the processing unit 620 through a user input interface 660 that is coupled to the system bus, but may be connected by other interface and bus structures, such as a parallel port, game port or a universal serial bus (USB). A monitor 691 or other type of display device is also connected to the system bus 621 via an interface, such as a video interface 690. In addition to the monitor, computers may also include other peripheral output devices such as speakers 697 and printer 696, which may be connected through a output peripheral interface 695.

The computer 610 may operate in a networked environment using logical connections to one or more remote computers, such as a remote computer 680. The remote computer 680 may be a personal computer, a server, a router, a network PC, a peer device or other common network node, and typically includes many or all of the elements described above relative to the computer 610, although only a memory storage device 681 has been illustrated in FIG. 10. The logical connections depicted in FIG. 10 include a local area network (LAN) 671 and a wide area network (WAN) 673, but may also include other networks. Such networking environments are commonplace in offices, enterprise-wide computer networks, intranets and the Internet.

When used in a LAN networking environment, the computer 610 is connected to the LAN 671 through a network interface or adapter 670. When used in a WAN networking environment, the computer 610 typically includes a modem 672 or other means for establishing communications over the WAN 673, such as the Internet. The modem 672, which may be internal or external, may be connected to the system bus 621 via the user input interface 660, or other appropriate mechanism. In a networked environment, program modules depicted relative to the computer 610, or portions thereof, may be stored in the remote memory storage device. By way of example, and not limitation, FIG. 10 illustrates remote application programs 685 as residing on memory device 681. It will be appreciated that the network connections shown are exemplary and other means of establishing a communications link between the computers may be used.

The various methods or processes outlined herein may be implemented in any suitable hardware. Additionally, the various methods or processes outlined herein may be implemented in a combination of hardware and of software executable on one or more processors that employ any one of a variety of operating systems or platforms. Examples of such approaches are described above. However, any suitable combination of hardware and software may be employed to realize any of the embodiments discussed herein.

Also, the various methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

In this respect, various inventive concepts may be embodied as at least one non-transitory computer readable storage medium (e.g., a computer memory, one or more floppy discs, compact discs, optical discs, magnetic tapes, flash memories, circuit configurations in Field Programmable Gate Arrays or other semiconductor devices, etc.) encoded with one or more programs that, when executed on one or more computers or other processors, implement the various embodiments of the present disclosure. The non-transitory computer-readable medium or media may be transportable, such that the program or programs stored thereon may be loaded onto any computer resource to implement various aspects of the present disclosure as discussed above.

The terms “program” or “software” are used herein in a generic sense to refer to any type of computer code or set of computer-executable instructions that can be employed to program a computer or other processor to implement various aspects of embodiments as discussed above. Additionally, it should be appreciated that according to one aspect, one or more computer programs that when executed perform methods of the present disclosure need not reside on a single computer or processor, but may be distributed in a modular fashion among different computers or processors to implement various aspects of the present disclosure.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

The embodiments described herein may be embodied as a method, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Further, some actions are described as taken by a “user.” It should be appreciated that a “user” need not be a single individual, and that in some embodiments, actions attributable to a “user” may be performed by a team of individuals and/or an individual in combination with computer-assisted tools or other mechanisms.

### Example 1

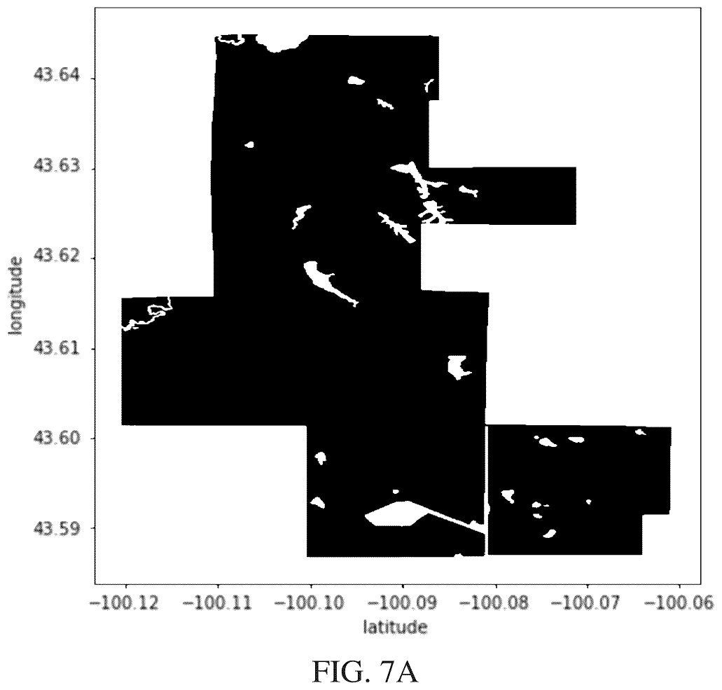

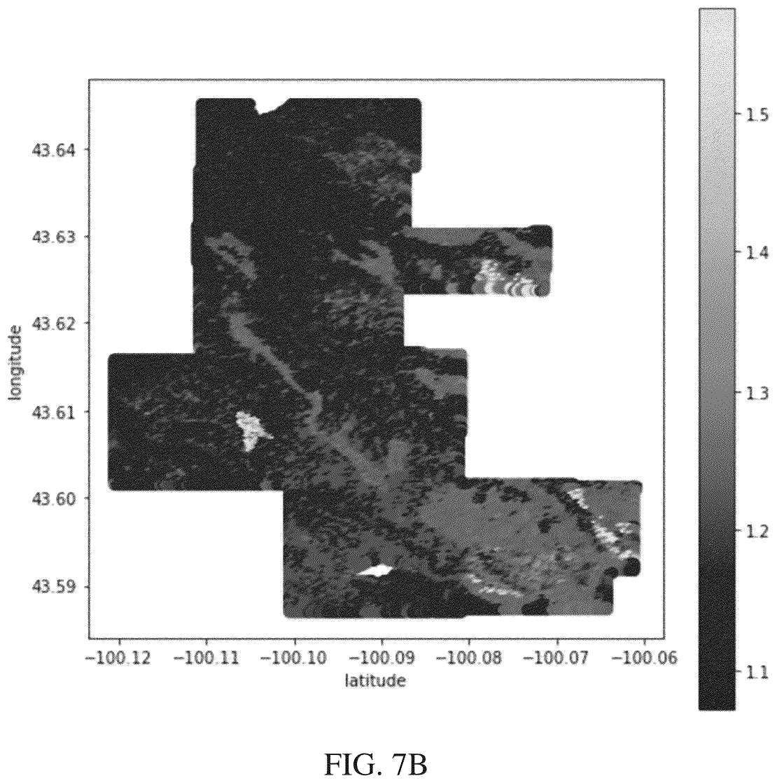

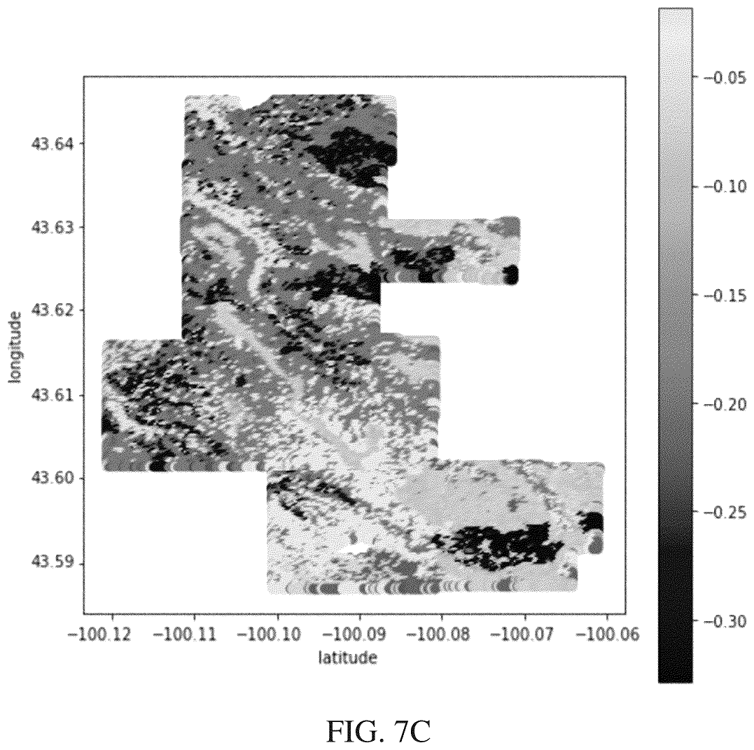

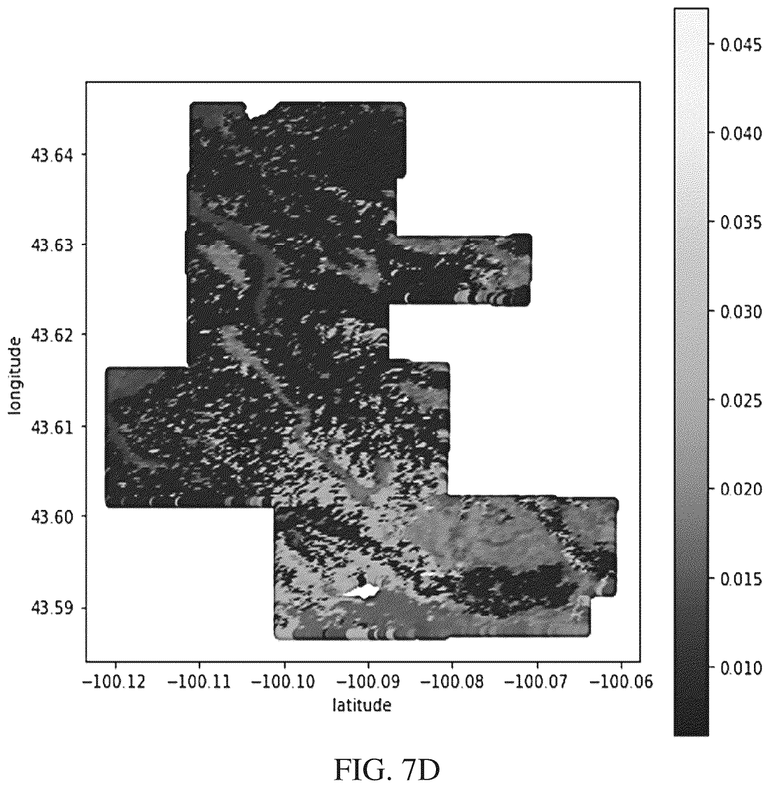

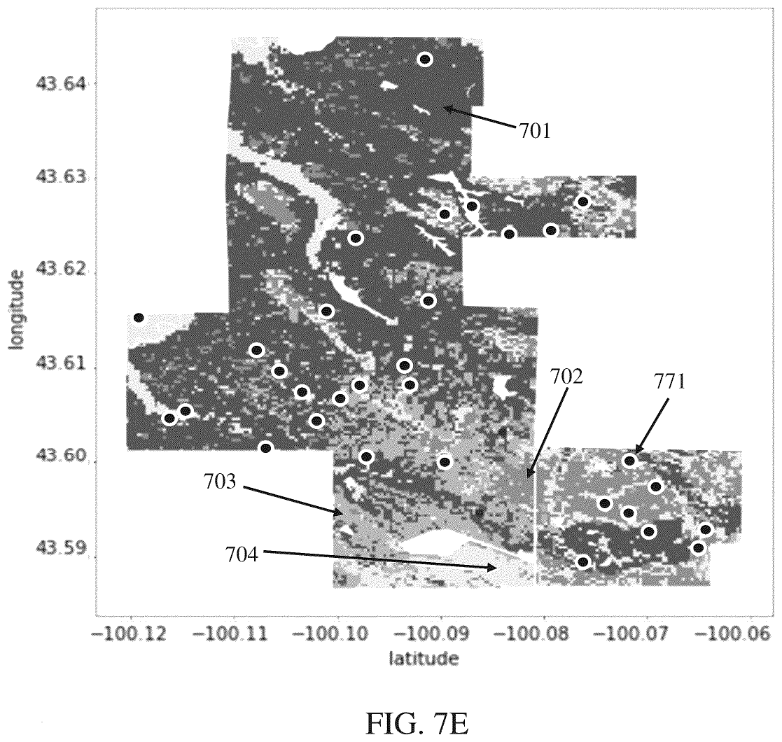

This example describes the stratification and sampling of an exemplary geographic region to determine a total carbon content of the geographic region. FIG. 7A shows the geographic region as a black cutout associated with the latitude and longitude presented on the X and Y axis, respectively. The geographic region was a single region with an area of 4154 acres and a depth of 5 ft. The geographic region was treated as an area (assuming a uniform carbon concentration as a function of depth) and was subdivided into 5 ft×5 ft pixels.

An initial dataset was obtained from the POLARIS database, which included soil properties associated with a tiling of the geographic region into 30 ft×30 ft pixels with a 5 cm depth. The 5 ft×5 ft pixels used for stratification of the geographic region thus subdivided the pixels of the Polaris database into 36 pixels used for stratification. Each stratification pixel was assigned a value of the soil properties that was randomly sampled from the distribution of the soil property for the POLARIS pixel that the stratification pixel overlapped, and the size of the stratification pixel was chosen to be 5 ft×5 ft to ensure that the stratification pixels subdividing each POLARIS pixel accurately sampled the statistical distribution of the POLARIS pixel.

FIG. 7B presents the bulk soil density of the POLARIS tiles for the geographic region, and FIG. 7C presents the standard deviation of soil density (which may be squared to determine the variance of soil density), in units of g/cm³. FIG. 7D presents POLARIS tiles representing the stock mean value of soil organic carbon content within the field, estimated from the organic matter content provided by POLARIS.

The stock mean value of soil organic carbon content shown in FIG. 7D was used to stratify the soil of the geographic region into a plurality of strata using k-means clustering. A target uncertainty was imposed by requiring a confidence level of 90% in the total carbon content of the geographic region. The geographic region was stratified into a plurality of groups of strata, and a minimum number of measurement locations required to achieve the target confidence level was determined to be 20 measurement locations, following a Neyman allocation to 4 strata. FIG. 7E presents the stratification of the geographic region into strata 701, 702, 703, and 704. Measurement locations 771 are also represented in FIG. 7E, as enlarged black circles with white borders.

Table 1 presents the number of measurement locations associated with each stratum, and shows the total carbon content of each stratum (tonnes) as well as the bulk carbon density (expressed in units of tonnes per pixel-volume), the number of pixels per stratum, and the area percentage of the geographic region associated with each stratum.

TABLE 1
Measured carbon content of the geographic area,
and properties of the various strata.

| | | | Carbon | Number | |
|---|---|---|---|---|---|
| | Measurement | Carbon | (tonnes | of | Area |
| Stratum | Locations | (tonnes) | per pixel) | Pixels | Percentage |
| 701 | 7 | 15477.337 | 0.039917 | 387738 | 57.7 |
| 702 | 4 | 10689.138 | 0.110852 | 96427 | 14.3 |
| 703 | 5 | 11078.511 | 0.136488 | 81168 | 12.1 |
| 704 | 4 | 9775.223 | 0.091428 | 106917 | 15.9 |
| Total | 20 | 47020.209 | N/A | 672250 | 100 |

This example demonstrates the viability of the approach described herein for the purpose of measuring bulk soil carbon content of a geographic region, and demonstrates its ability to accurately sample large geographic regions using relatively small numbers of measurement locations.

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art. Accordingly, the foregoing description and drawings are by way of example only.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

1. A method of determining bulk soil carbon content in a geographic region, the method comprising:
obtaining an initial dataset including at least one soil property related to bulk soil carbon content for the geographic region;
clustering a plurality of tiles corresponding to subdivided portions of the geographic region based at least in part on the at least one soil property to form strata; and
determining measurement locations within the strata to be sampled to determine the bulk soil carbon content of the geographic region.

2. The method of claim 1, further comprising actually determining the bulk soil carbon content of the geographic region.

3. The method of claim 1, further comprising outputting the measurement locations to a user.

4. The method of claim 1, further comprising storing the measurement locations on non-transitory computer readable memory.

5. The method of claim 1, further comprising:
obtaining a target uncertainty for bulk soil carbon content;
performing the clustering step for a range of different numbers of strata to generate a plurality of groups of strata, wherein each group includes a different number of strata; and
for each group of strata, determining a minimum number of measurement locations to determine the bulk soil carbon content of the geographic region with an uncertainty within the target uncertainty; and
identifying the group of strata with a smallest minimum number of measurement locations;
wherein determining the measurement locations includes determining the measurement locations within the strata of the identified group of strata.

6. The method of claim 1, further comprising:
obtaining a target number measurement locations;
performing the clustering step for a range of different numbers of strata to generate a plurality of groups of strata, wherein each group includes a different number of strata;
for each group of strata, determining a minimum uncertainty with which the bulk soil carbon content of the geographic region can be determined using the target number of measurement locations; and
identifying the group of strata with a smallest minimum uncertainty;
wherein determining the measurement locations includes determining the measurement locations within the strata of the identified group of strata.

7. The method of claim 1, further comprising using a Neyman allocation to identify an allocation of measurement locations between the strata.

8. The method of claim 1, further comprising measuring bulk soil carbon content at one or more measurement locations of the set of measurement locations.

9. The method of claim 1, further comprising determining values of the soil property for the plurality of tiles by determining a statistical distribution of the soil property from the dataset and randomly assigning the values of the soil property to the tiles based on the statistical distribution of the soil property.

10. The method of claim 9, further comprising artificially increasing variance of the statistical distribution prior to assigning the values of the soil properties of the tiles.

11. The method of claim 1, wherein the plurality of tiles is a first plurality of tiles, and the initial dataset includes the at least one soil property for a second plurality of tiles corresponding to subdivided portions of the geographic region.

12. The method of claim 11, further comprising mapping the second plurality of tiles onto the first plurality of tiles.

13. The method of claim 9, wherein the random assignment of values of the soil property to a tile of the first plurality of tiles is based on a statistical distribution of the soil property determined from one or more tiles of the second plurality of tiles that overlaps the tile of the first plurality.

14. The method of claim 11, wherein the first plurality of tiles is a subdivision of the second plurality of tiles.

15. The method of claim 1, wherein determining measurement locations within the strata includes determining measurement locations within each stratum of the strata.

16. A soil measurement planning system comprising:
a processor configured to:
obtain an initial dataset including at least one soil property related to bulk soil carbon content for the geographic region;
cluster a plurality of tiles corresponding to subdivided portions of the geographic region based at least in part on the at least one soil property to form strata; and
determine measurement locations within the strata to be sampled to determine the bulk soil carbon content of the geographic region.

17. The soil measurement planning system of claim 16, further configured to output the measurement locations to a user.

18. The soil measurement planning system of claim 16, further configured to store the sampling plan on non-transitory computer readable memory.

19. The soil measurement planning system of claim 16, further configured to:
obtain a target uncertainty for bulk soil carbon content;
perform the clustering step for a range of different numbers of strata to generate a plurality of groups of strata, wherein each group includes a different number of strata;
for each group of strata, determine a minimum number of measurement locations to determine the bulk soil carbon content of the geographic region with a uncertainty within the target uncertainty; and
identify the group of strata with the smallest minimum number of measurement locations;
wherein determining the measurement locations includes determining the measurement locations within the strata of the identified group of strata.

20. The soil measurement planning system of claim 16, further configured to:
obtain a target number measurement locations;
perform the clustering step for a range of different numbers of strata to generate a plurality of groups of strata, wherein each group includes a different number of strata;
for each group of strata, determine a minimum uncertainty with which the bulk soil carbon content of the geographic region can be determined using the target number of measurement locations; and
identify a group of strata with the smallest minimum uncertainty;
wherein determining the measurement locations includes determining the measurement locations within the strata of the identified group of strata.

21. The soil measurement planning system of claim 16, further configured to use a Neyman allocation to identify an allocation of measurement locations between the strata.

22-28. (canceled)

29. The method of claim 1, wherein the bulk soil carbon content is a bulk soil carbon density or a total soil carbon content.

30. (canceled)

31. The method of claim 1, wherein obtaining the initial dataset comprises downloading the initial dataset from a remotely located server, recalling the initial dataset from non-transitory computer readable memory, or collecting and storing the initial dataset from preliminary measurements of the geographic region.

32-44. (canceled)

45. The method of claim 1, wherein the set of measurement locations does not include measurement locations within a buffer zone extending around an outer boundary of the geographic region.

46-47. (canceled)

48. At least one non-transitory computer-readable storage medium storing processor executable instructions that, when executed by at least one processor, cause the at least one processor to perform the method of claim 1.
